(19) United States
(12) Patent Application Publication
(10) Pub. No.: US 2026/0232934 A1
(43) Pub. Date: Aug. 13, 2026

(54) CONTROL OF A BREATHING ASSISTANCE APPARATUS

(71) Applicant: Fisher & Paykel Healthcare Limited, Auckland (NZ)

(72) Inventors: Luca ORTALI, Auckland (NZ); Jae Chul HAN, Auckland (NZ); Samuel Robertson FRAME, Auckland (NZ)

(21) Appl. No.: 19/155,493

(22) PCT Filed: Feb. 12, 2024
(86) PCT No.: PCT/IB2024/051272

§ 371 Date: Aug. 11, 2025

Related U.S. Application Data
(60) Provisional application No. 63/484,690, filed on Feb. 13, 2023.

(51) Int. Cl. A61M 16/00; A61M 16/16; H01M 10/42

(52) U.S. Cl. CPC A61M 16/0003; A61M 16/024; A61M 16/16; H01M 10/425; A61M 2016/0027; A61M 2016/0039; A61M 2205/3317; A61M 2205/3561; A61M 2205/505; A61M 2205/8206

(57) ABSTRACT

A breathing assistance apparatus is disclosed. The apparatus may include a battery and a controller. The battery may be configured to supply power to at least one component of the apparatus. The at least one component may be one or more of a humidifier heater, a flow generator, or a conduit: the conduit may include a conduit heater. The controller may be configured to control the at least one component of the apparatus to discharge the battery to a predetermined charge level.

### FIELD OF TECHNOLOGY

The present disclosure relates to control of a battery discharge of breathing assistance apparatuses.

### BACKGROUND

Breathing assistance apparatuses are used to deliver a flow of gas to patients in various environments (such as hospital, medical facility, residential care, or home environments). A breathing assistance apparatus (e.g. a flow therapy apparatus) may include an oxygen inlet that enables the respiratory apparatus to deliver supplemental oxygen with the flow of gas. A breathing assistance apparatus may also (or alternatively) include a humidification apparatus that enables the respiratory apparatus to deliver heated and humidified gases. A breathing assistance apparatus may allow adjustment of, and control over, characteristics of the gases flow. These characteristics may include for example flow rate, temperature, gas concentration (such as supplemental oxygen concentration), humidity, and pressure, etc.

Patients suffering from various health conditions and diseases can benefit from breathing assistance (for example respiratory therapy). In at least one form, the respiratory therapy may be oxygen therapy. For example, a patient suffering from chronic obstructive pulmonary disease (COPD), pneumonia, asthma, bronchopulmonary dysplasia, heart failure, cystic fibrosis, sleep apnea, lung disease, trauma to the respiratory system, acute respiratory distress, and/or other conditions or diseases can benefit from respiratory therapy. Similarly, patients receiving pre- and post-operative oxygen delivery can also benefit from respiratory therapy.

### SUMMARY

In a first aspect, the present invention may be said to broadly consist in a breathing assistance apparatus comprising:
a battery configured to supply power to at least one component of the apparatus,
the at least one component comprising one or more of:
   a humidifier heater,
   a flow generator,
   a conduit, the conduit comprising a conduit heater,
a controller configured to control the at least one component of the apparatus to discharge the battery to a predetermined charge level.

In some configurations, the controller is in communication with the battery, and wherein the controller is configured to determine one or more of: a state of charge of the battery, an electrical characteristic of the battery, a chemical characteristic of the battery and/or a physical characteristic of the battery.

In some configurations, the controller is provided with one or more of: a state of charge of the battery, an electrical characteristic of the battery, a chemical characteristic of the battery and/or a physical characteristic of the battery.

In some configurations, the battery comprises a battery controller in communication with the controller of the apparatus, and wherein the battery controller is configured to determine one or more of: a state of charge of the battery, an electrical characteristic of the battery, a chemical characteristic of the battery and/or a physical characteristic of the battery.

In some configurations, the predetermined charge level is an electrical characteristic (optionally a voltage).

In some configurations, the electrical characteristic is indicative of a state of charge of the battery.

In some configurations, the predetermined charge level is indicative of a state of charge of the battery.

In some configurations, the predetermined charge level is a predetermined state of charge, and wherein the controller is configured to control discharge the battery to the predetermined state of charge.

In some configurations, the predetermined charge level is based on, at least in part, at least one battery discharge configuration.

In some configurations, the at least one battery discharge configuration comprises a shipping configuration, wherein in the shipping configuration the predetermined charge level is a shipping configuration charge level.

In some configurations, the shipping configuration charge level is about a 30% state of charge).

In some configurations, the at least one battery discharge configuration comprises a storage configuration, wherein in the storage configuration the predetermined charge level is a storage configuration charge level.

In some configurations, the storage configuration charge level is about a 60% state of charge.

In some configurations, in the storage configuration the predetermined charge level is based on an intended duration of storage.

In some configurations, when the intended duration of storage is about 6 months to about 12 months, the predetermined charge level is a first storage configuration charge level In some configurations, the first storage configuration charge level is about a 30% state of charge.

In some configurations, when the intended duration of storage is greater than about 12 months, the predetermined charge level is a second storage configuration charge level.

In some configurations, the second storage configuration charge level is about a 60% state of charge.

In some configurations, a user can enter the intended storage duration via a user interface.

In some configurations, when a user selects the storage configuration, the apparatus prompts (optionally via a display) to enter the intended storage duration via a user interface.

In some configurations, the at least one battery discharge configuration comprises a recall configuration, wherein in the recall configuration the predetermined charge level is a recall configuration charge level.

In some configurations, the recall configuration charge level about a 10% state of charge.

In some configurations, the at least one battery discharge configuration comprises a disposal configuration, wherein in the disposal configuration the predetermined charge level is a disposal configuration charge level.

In some configurations, the disposal configuration charge level is about a 0% state of charge.

In some configurations, the controller is configured to control the at least one component of the apparatus to discharge the battery at a predetermined discharge rate.

In some configurations, the predetermined discharge rate is a C-rate.

In some configurations, the predetermined discharge rate is based on the battery discharge configuration.

In some configurations, when the at least one battery discharge configuration is a or the storage configuration or a or the shipping configuration the predetermined discharge rate is a first discharge rate.

In some configurations, when the at least one battery discharge configuration is a recall configuration or a disposal configuration the predetermined discharge rate is a second discharge rate.

In some configurations, the second discharge rate is lower than the first discharge rate.

In some configurations, the controller is configured to control discharge of the battery to control a charge level of the battery to the predetermined charge level.

In some configurations, the charge level is indicative of a state of charge of the battery.

In some configurations, the charge level of the battery is based on one or more of:
an electrical characteristic of the battery,
a chemical characteristic of the battery,
a physical characteristic of the battery.

In some configurations, a determined charge level indicative of the charge level of the battery is configured to be continuously, or periodically updated.

In some configurations, controlling the at least one component comprises one or more of:
activating and/or deactivating the at least one component
controlling a power draw of the at least one component,
controlling an output of the at least one component.

In some configurations, controlling the at least one component is based on the battery discharge configuration.

In some configurations, the controller is configured to:
activate the at least one component, increase the power draw of the at least one component, and/or increase the output of the at least one component, and then,
deactivate the at least one component, decrease the power draw of the at least one component, and/or decrease the output of the at least one component,
optionally the controller may undertake the activating and/or increasing and deactivating and/or decreasing repeatedly.

In some configurations, after the controller activates the at least one component, increases the power draw of the at least one component, and/or increases the output of the at least one component, the controller is configured to maintain the output of the at least one component (optionally for a predetermined amount of time).

In some configurations, after the controller deactivates the at least one component, decreases the power draw of the at least one component, and/or decreases the output of the at least one component the controller is configured to maintain the output of the at least one component (optionally for a predetermined amount of time).

In some configurations, the controller is configured to control an output of the flow generator to discharge the battery to the predetermined charge level.

In some configurations, the flow generator comprises a blower, the blower including a motor.

In some configurations, the output of the flow generator is one or more of: a speed of the motor, a flow rate of gases provided by the flow generator, a pressure provided by the flow generator.

In some configurations, the controller is configured to control the output of the flow generator to:
achieve a predetermined output of the flow generator, or
be within a predetermined output range of the flow generator, or
be above a predetermined output of the flow generator.

In some configurations, the predetermined output is greater than a therapeutic output of the flow generator, the therapeutic output being an output when the apparatus is providing therapy to a user.

In some configurations, the predetermined output range of the flow generator is a motor speed range of about 5,000 RPM to about 20,000 RPM, or about 23,000 RPM to about 26,000 RPM.

In some configurations, the predetermined output is a motor speed of about 20,000 RPM.

In some configurations, the controller is configured to control the output of the flow generator to repeatedly (optionally periodically) increase and decrease the output of the flow generator (optionally within a predetermined output range of the flow generator).

In some configurations, subsequent to the controller increasing the output of the flow generator, the controller is configured to maintain the output of the flow generator (optionally for a predetermined amount of time) before subsequently decreasing the output of the flow generator.

In some configurations, subsequent to the controller decreasing the output of the flow generator, the controller is configured to maintain the output of the flow generator (optionally for a predetermined amount of time) before subsequently increasing the output of the flow generator.

In some configurations, the controller is configured to control the output of the flow generator to increase the output of the flow generator to a:
first predetermined flow generator output,
be within a first predetermined flow generator output range, or
be above a first predetermined flow generator output, and subsequently decrease the output of the flow generator to:
a second predetermined flow generator output,
be within a second predetermined flow generator output range, or
be below a second predetermined flow generator output.

In some configurations, decreasing the output of the flow generator comprises allowing the motor of the flow generator to freewheel or controlling the output of the flow generator to decrease.

In some configurations, decreasing the flow generator comprises a braking system configured to brake the motor of the flow generator.

In some configurations, the braking system is configured to decrease the output of the flow generator (optionally by decreasing the motor speed).

In some configurations, the controller is configured to control a power delivered to the humidifier heater and/or the conduit heater.

In some configurations, the power delivered to the humidifier heater and/or the conduit heater is controlled by regulating a current or voltage delivered to the humidifier heater and/or the conduit heater.

In some configurations, the power delivered to the humidifier heater and/or the conduit heater is controlled by a digital control scheme.

In some configurations, the controller is configured to control the humidifier heater and/or the conduit heater via pulse width modulation.

In some configurations, the controller is configured to provide a signal to the humidifier heater and/or the conduit heater to control the power delivered to the humidifier heater and/or the conduit heater.

In some configurations, the controller is configured to control the power delivered to the humidifier heater and/or the conduit heater to discharge the battery to the predetermined charge level.

In some configurations, the controller is configured to control the power delivered to the humidifier heater and/or the conduit heater to achieve a predetermined output of the humidifier heater and/or the conduit heater.

In some configurations, the controller is configured to control the power delivered to the humidifier heater and/or the conduit heater to achieve a predetermined heater power, or be within a predetermined heater power range, or be above a predetermined heater power.

In some configurations, the predetermined heater power is greater than a therapeutic power, the therapeutic power being a power of the heater when the apparatus is providing therapy to a user.

In some configurations, the predetermined heater power corresponds with a safe threshold for a user.

In some configurations, the controller is configured to control the power delivered to the humidifier heater and/or the power delivered to the conduit heater to repeatedly (optionally periodically) increase and decrease the power delivered to the humidifier heater and/or the power delivered conduit heater (optionally within a gas target temperature range and/or a target humidifier heater temperature range and/or a target conduit heater temperature range).

In some configurations, subsequent to the controller increasing the power delivered to the humidifier heater and/or the power delivered to the conduit heater, the controller is configured to maintain the power delivered to the humidifier heater and/or the power delivered conduit heater before subsequently decreasing the power delivered to the humidifier heater and/or the power delivered to the conduit heater.

In some configurations, subsequent to the controller decreasing the power delivered to the humidifier heater and/or the power delivered to the conduit heater, the controller is configured to maintain the power delivered to the humidifier heater and/or the power delivered conduit heater (optionally for a predetermined amount of time) before subsequently increasing the power delivered to the humidifier heater and/or the power delivered to the conduit heater.

In some configurations, the controller is configured to control the power delivered to the humidifier heater and/or the power delivered to the conduit heater to increase the power delivered to the humidifier heater and/or the power delivered to the conduit heater to:

a first predetermined power delivered to the humidifier heater and/or a first predetermined power delivered to the conduit heater,
within a first predetermined power delivered to the humidifier heater range and/or within a first predetermined power delivered to the conduit heater range, or
above a first predetermined power delivered to the humidifier heater and/or above a first predetermined power delivered to the conduit heater, and

subsequently decrease the power delivered to the humidifier heater and/or the power delivered to the conduit heater to:
a second predetermined power delivered to the humidifier heater and/or a second predetermined power delivered to the conduit heater,
within a second predetermined power delivered to the humidifier heater range and/or within a second predetermined power delivered to the conduit heater range, or
below a second predetermined power delivered to the humidifier heater and/or a below a second predetermined power delivered to the conduit heater.

In some configurations, the controller is configured to control the power delivered to the humidifier heater and/or the conduit heater to achieve a target gas temperature, wherein the target gas temperature is a temperature of the gases at one or more of locations, the one or more locations comprising:
an inlet of the humidifier,
an outlet of the humidifier,
a location in the conduit,
a location at the patient end of the conduit.

In some configurations, the apparatus comprises at least one temperature sensor, the at least one temperature sensor configured to measure the temperature of the gases at the one or more locations.

In some configurations, the target gas temperature is greater than a therapeutic gas temperature when the apparatus is providing therapy to a user.

In some configurations, the target gas temperature corresponds with a safe threshold for a user.

In some configurations, the controller is configured to control the power delivered to the humidifier heater conduit to achieve a target humidifier heater temperature and/or to control the power delivered to the conduit heater to achieve a target conduit heater temperature, optionally the target humidifier heater temperature and/or the target conduit heater temperature is less than about 60 degrees Celsius.

In some configurations, the controller is configured to control the power delivered to the humidifier heater to achieve a target humidifier heater temperature range and/or to control power delivered to the conduit heater to achieve a target conduit heater temperature range, wherein the target humidifier heater temperature range and/or the target conduit heater temperature range is about 40 degrees Celsius to about 60 degrees Celsius.

In some configurations, the apparatus comprises at least one heater temperature sensor, the at least one heater temperature sensor configured to measure a temperature of the humidifier heater and/or a temperature conduit heater.

In some configurations, the temperature of the humidifier heater and/or the temperature of the conduit heater is/are greater than a therapeutic heater temperature when the apparatus is providing therapy to a user.

In some configurations, the temperature of the humidifier heater and/or the temperature of the conduit heater correspond(s) with a safe threshold for a user.

In some configurations, the controller is configured to control a or the target gas temperature and/or a or the target humidifier heater temperature and/or a or the target conduit heater temperature to repeatedly (optionally periodically) increase and decrease (optionally within a or the gas target temperature range and/or a or the target humidity heater temperature range and/or a or the target conduit heater temperature range).

In some configurations, subsequent to the controller increasing the target gas temperature and/or the target humidifier heater temperature and/or the target conduit heater temperature, the controller is configured to maintain the target gas temperature and/or the target humidifier heater temperature and/or the target conduit heater temperature before subsequently decreasing the target gas temperature and/or the target humidifier heater temperature and/or the target conduit heater temperature.

In some configurations, subsequent to the controller decreasing the target gas temperature and/or the target humidifier heater temperature and/or the target conduit heater temperature, the controller is configured to maintain the target gas temperature and/or the target humidifier heater temperature and/or the target conduit heater temperature (optionally for a predetermined amount of time) before subsequently increasing the target gas temperature and/or the target humidifier heater temperature and/or the target conduit heater temperature.

In some configurations, the controller is configured to control the target gas temperature and/or the target heater temperature to increase the target gas temperature and/or the target heater temperature to a:
first predetermined target gas temperature and/or first predetermined target humidifier heater temperature and/or a first predetermined target conduit heater temperature,
within a first predetermined target gas temperature range and/or within a first predetermined target humidifier heater temperature range and/or within a first predetermined target conduit heater temperature range, or
above a first target gas temperature and/or above a first target humidifier heater temperature and/or above a first target conduit heater temperature, and

subsequently decrease the output of the flow generator to:
a second predetermined target gas temperature and/or a second predetermined target humidifier heater temperature and/or a second predetermined target conduit heater temperature,
within a second predetermined target gas temperature range and/or within a second predetermined target humidifier heater temperature range and/or within a second predetermined target conduit heater temperature range, or
below a second target gas temperature and/or below a second target humidifier heater temperature and/or below a second target conduit heater temperature.

In some configurations, the controller is configured to power off the apparatus once the battery is discharged to a or the predetermined charge level.

In some configurations, the apparatus comprises at least one display, the display being in communication in with controller.

In some configurations, the controller is configured to control the at least one display to display a notification that the battery is being discharged.

In some configurations, the controller is configured to control the at least one display to display the battery discharge configuration.

In some configurations, the apparatus comprises a user interface (optionally the user interface is a touch interface as part of the at least one display).

In some configurations, a user can initiate the battery discharging to a or the predetermined charge level via the user interface.

In some configurations, the apparatus is configured to allow the user to initiate the battery discharging to a or the predetermined charge level based on one or more of:
the user providing credentials associated with permissions allowing the initiation of the battery discharging to a or the predetermined charge level;
accessing a service menu, optionally via providing a sequence of inputs via the user interface.

In some configurations, the apparatus is configured to allow the user to initiate the battery discharging to a or the predetermined charge level based on a message from a remote device.

In some configurations, a user can cancel the battery discharging via the user interface.

In some configurations, the at least one display comprises an interactable element configured to trigger the controller to cancel the battery discharging.

In some configurations, the apparatus is configured to determine whether a patient is connected to a patient interface.

In some configurations, the apparatus is configured to determine whether a patient is connected to the patient interface based on a flow rate and/or a pressure data of the flow of gases.

In some configurations, the apparatus is configured to determine whether a patient is connected to the patient interface based on a resistance to flow of the flow of gases.

In some configurations, when the apparatus determined a patient is connected to the patient interface, the controller is configured to cancel the battery discharging.

In some configurations, the humidifier is configured to be pneumatically connected to the flow generator and to humidify the flow of gases generated by the flow generator.

In some configurations, the conduit is connected to an outlet of the apparatus.

In some configurations, the conduit is one or more of: an expiratory conduit, an inspiratory conduit, a patient supply conduit.

In some configurations, the conduit is pneumatically connected to the apparatus.

In some configurations, the conduit forms part of a gases flow path.

In some configurations, the apparatus comprises a heater plate as the humidifier heater, wherein the heater plate is configured to heat the contents of a humidification chamber.

In some configurations, the controller is configured to control the at least one component of the apparatus to discharge the battery to a or the predetermined charge level when the apparatus receives a message from a device.

In some configurations, the apparatus comprises at least one communications module configured to receive the message from the device.

In some configurations, the device is a one or more of:
a server,
a computer,
a phone,
a tablet.

In some configurations, the device is configured to select one of a plurality of connected apparatuses by a device ID associated with each apparatus.

In some configurations, the device is configured to receive an input from a or the user interface and based on the input communicate the message to the apparatus.

In another aspect there is provided a method of preparing a battery of a breathing assistance apparatus comprising:
controlling the at least one component of the apparatus to discharge the battery to a predetermined charge level, wherein the at least one component comprises:
a heater of a humidifier,
   a flow generator,
   a conduit configured to be connected to the apparatus, the conduit comprising a heater.

In another aspect there is provided a breathing assistance system comprising:
a flow generator,
a humidifier configured to be pneumatically connected to the flow generator and to humidify a flow of breathable gases generated by the flow generator, the humidifier comprising at least one heater (optionally the heater is a heater plate),
a conduit configured to be powered by the apparatus, the conduit comprising a heater,
a controller configured to control one or more of: the flow generator, the humidifier heater, and/or the conduit heater, to discharge the battery to a predetermined charge level.

In another aspect there is provided a breathing assistance apparatus comprising:
a battery configured to supply power to at least one component of the apparatus,
the at least one component comprising one or more of:
   a heater of a humidifier,
   a flow generator,
   a conduit configured to be powered by the apparatus, the conduit comprising a heater,
a controller configured to control the at least one component of the apparatus to discharge the battery to a predetermined charge level based on the battery discharge configuration,
the controller configured to receive a command relating to the selection of a battery discharge configuration from a user interface, wherein the user interface is part of the apparatus, or another device.

As used herein the term “and/or” means “and” or “or”, or both.

As used herein “(s)” following a noun means the plural and/or singular forms of the noun.

For the purposes of this specification, the term “plastic” shall be construed to mean a general term for a wide range of synthetic or semisynthetic polymerization products, and generally consisting of a hydrocarbon-based polymer.

For the purpose of this specification, where method steps are described in sequence, the sequence does not necessarily mean that the steps are to be chronologically ordered in that sequence, unless there is no other logical manner of interpreting the sequence.

The term “comprising” as used in the specification and claims means “consisting at least in part of.” When interpreting each statement in this specification that includes the term “comprising,” features other than that or those prefaced by the term may also be present. Related terms “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other aspects of the invention may become apparent from the following description which is given by way of example only and with reference to the accompanying drawings.

### BRIEF DESCRIPTION OF THE DRAWINGS

Preferred embodiments of the invention will be described by way of example only and with reference to the drawings, in which:

FIG. 1 shows in diagrammatic form a breathing assistance apparatus.

FIGS. 2, 3A and 3B show perspective views of a breathing assistance apparatus.

FIG. 4 shows a rear perspective view of a breathing assistance apparatus.

FIG. 5 shows a schematic gas flow path diagram for the filter module and the valve module, with the solid line arrows representing the flow of gases.

FIG. 6 shows a flow diagram illustrating entering a shipping configuration.

FIG. 7 shows a flow diagram illustrating entering a storage configuration.

FIG. 8 shows a flow diagram illustrating prompting a user for input for entering a storage configuration.

FIG. 9 shows a flow diagram illustrating entering a recall configuration.

FIG. 10 shows a flow diagram illustrating entering a disposal configuration.

FIG. 11 shows a flow diagram illustrating entering a battery discharge configuration and discharging the battery to a predetermined level.

FIG. 11A shows a flow diagram illustrating activating, increasing the power output and/or increasing the output of a component and then deactivating, decreasing the power output, and/or decreasing the output of the component.

FIG. 11B shows a flow diagram illustrating activating, increasing the power output and/or increasing the output of a component, maintaining, and then deactivating, decreasing the power output, and/or decreasing the output of the component and the reverse.

FIG. 12 shows a flow diagram illustrating increasing and decreasing the output of a flow generator.

FIG. 12A shows a flow diagram illustrating increasing the output of a flow generator, maintaining, and then decreasing the output of a flow generator and the reverse.

FIG. 13 shows a flow diagram illustrating increasing and decreasing the power delivered to the heater of the humidifier and/or the heater of the conduit.

FIG. 13A shows a flow diagram illustrating increasing the power delivered to the heater of the humidifier and/or the heater of the conduit, maintaining, and then decreasing the power delivered to the heater of the humidifier and/or the heater of the conduit and the reverse.

FIG. 14 shows a flow diagram illustrating increasing and decreasing the target gas temperature and/or the target heater temperature.

FIG. 14A shows a flow diagram illustrating increasing the target gas temperature and/or the target heater temperature, maintaining, and then decreasing the target gas temperature and/or the target heater temperature and the reverse.

FIG. 15 shows an example of a display showing options for a user to select different battery discharge configuration.

FIG. 15A shows an example of a discharge menu shown on the display during the discharge process.

FIG. 16 shows a flow diagram illustrating receiving a message from a device and controlling a component to discharge the battery to a predetermined level.

FIG. 17 shows a flow diagram illustrating a communications module receiving a message from a device and controlling a component to discharge the battery to a predetermined level.

### DETAILED DESCRIPTION OF PREFERRED EMBODIMENTS

A breathing assistance apparatus 10 provides therapy to a user for example any one or combination of: Nasal High Flow (NHF) therapy, Continuous Positive Airway Pressure (CPAP) therapy, Non-Invasive Ventilation (NIV) and Bubble Continuous Positive Airway Pressure (BCPAP) therapy.

During operation the apparatus 10 may operate from a number of power sources. In some cases, the apparatus 10 may be powered by a mains power supply (for example from the power grid). In some cases, the apparatus 10 may be powered by a battery. In some embodiments the battery may be a plurality of batteries. In some embodiments, the battery may be provided as part of a battery pack.

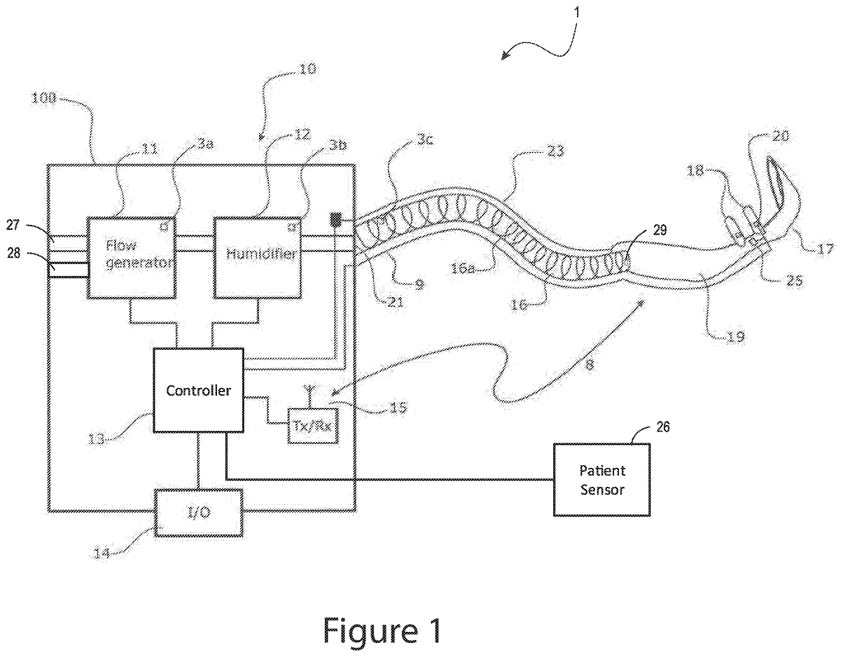

A breathing assistance apparatus 10 is shown in FIG. 1. The breathing assistance apparatus 10 can comprise a housing 100 (for example as a single housing) that contains one or more of: a flow generator 11, which in some embodiments is in the form of a motor/impeller arrangement (for example, a blower), a humidifier 12 pneumatically connected to the flow generator 11, a controller 13, and a user interface 14 (comprising, for example, a display and input device(s) such as button(s), a touch screen, or the like).

In FIG. 1, the apparatus 10 shows a housing 100 comprising a flow generator 11 and a humidifier 12 pneumatically connected to the flow generator 11. As shown in FIG. 1 the humidifier 12 and flow generator 11 are integrated into a common housing. This provides a compact device that can be easily moved around or carried to provide mobility. Further the flow generator 11 and humidifier 12 being combined in the same housing allows for simpler set up (i.e., chamber 300 can be positioned in the housing or a dock provided by the housing).

In some embodiments, the breathing assistance apparatus 10 may not comprise a flow generator 11. In this case the apparatus 10 does not generate a flow of gases, and instead is configured to be connected to an external flow generator and configured to humidify the flow of gases from the external flow generator. For example, the breathing assistance apparatus 10 can be used as a stand-alone humidifier to humidify gases flowing through the humidifier. The flow generator may be wall gas or a ventilator or other separate flow generator that can be configured to provide one of the therapies described elsewhere in the specification (e.g. NIV, NHF, CPAP, BCPAP, invasive ventilation etc). The humidifier may include a battery coupled to the humidifier to supply power when mains is unavailable (as described in more detail below). In some embodiments, the battery may be removably coupled to the apparatus and is rechargeable. The humidifier is pneumatically coupled to a flow generator via a conduit and a separate conduit couples to the humidifier to convey humidified gases from the humidifier to a patient.

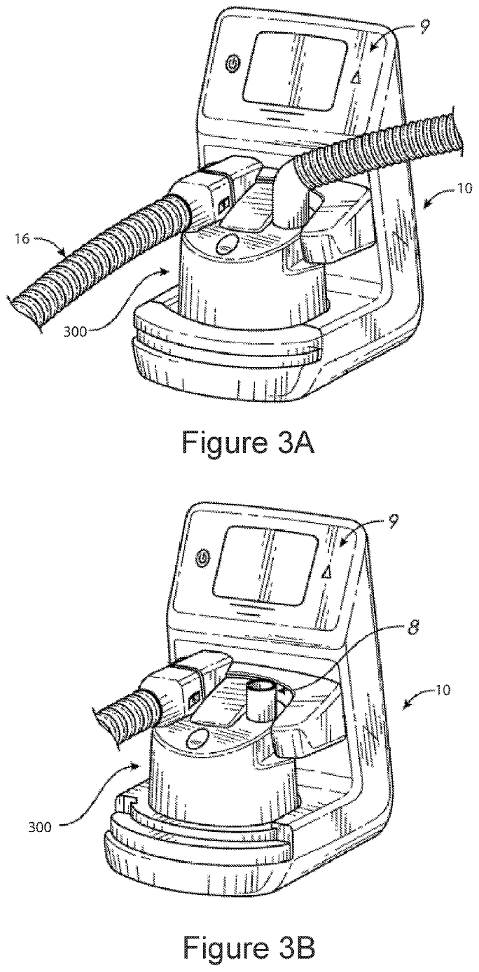

An example of an apparatus 10 as a humidifier (i.e., without a flow generator) is shown in FIGS. 3A and 3B. The apparatus includes a connector that pneumatically connects a conduit 16 (as described in more detail above) to an outlet (as a gases outlet of the apparatus) of a humidification chamber 31. The conduit 16 may be an inspiratory limb of a patient circuit, i.e., configured to deliver humidified gases to a user, such as via a patient interface (not shown). The conduit 16 may have a conduit heater 16a (as for example described elsewhere in the specification).

An inlet 8 of the humidification chamber 300 is configured to be fluidly connected to a flow generator positioned remote from the apparatus 10 (for example by the conduit shown connected to inlet 8 in FIG. 3A).

As shown in FIGS. 3A and 3B the apparatus 10 further includes a panel 9 which may be used to mount a user display and/or controls. For example, various dials, switches, and other input means may be used to control operation of the device. Additionally, or alternatively, a touch screen display may be used. The user display may display parameters of the system, warnings in the event of any errors or malfunctions, or prompts where user action is required, etc. Where a touch screen display is used, the same display may be used to present information to a user and receive inputs from a user, at least in part (as for example described elsewhere in the specification). The humidifier apparatus shown in FIGS. 3A and 3B may comprise multiple sensors. For example, the humidifier 10 may comprise a flow sensor, one or more temperature sensors, one or more pressure sensors and one or more humidity sensors. In one example configuration the humidifier comprises at least a temperature sensor positioned within or adjacent the inlet and a temperature sensor within or adjacent the outlet of the chamber 300. Optionally the humidifier may comprise a flow sensor within or adjacent the outlet of the chamber 300. Additionally, a further flow sensor may be located within or adjacent the inlet 8. Optionally, the humidifier may include a single flow sensor located in the inlet or outlet. The humidifier may additionally comprise one or more humidity sensors, that may be arranged within or adjacent either of the inlet or outlet, or in the inlet and outlet. Further, there may be additional ambient temperature sensors. The humidifier may also comprise additional sensors associated with the heater e.g., temperature sensors associated with the heater.

In some configurations, the apparatus 10 as shown in FIGS. 3A and 3B may comprise a battery (as discussed in more detail below.) The apparatus 10 may for example be the apparatus as described in WO2015/093989 which is incorporated by reference in its entirety.

The humidifier 12 can humidify the gases flow and/or heat the gases flow to an appropriate level. The controller 13 can be configured to control the humidifier 12 (for example by controlling at least a humidifier heater).

The humidifier 12 may comprise a humidification chamber. The humidification chamber may be configured to be removed from the humidifier (for example for replacement, cleaning and/or refilling). Alternatively, the humidification chamber may be non-removable from the humidifier.

The humidification chamber may comprise an autofill mechanism that comprises at least a valve and a float coupled to the valve. The humidification chamber in use can be coupled to water reservoir of water bag to auto fill. Alternatively, the humidification chamber may be manually refilled.

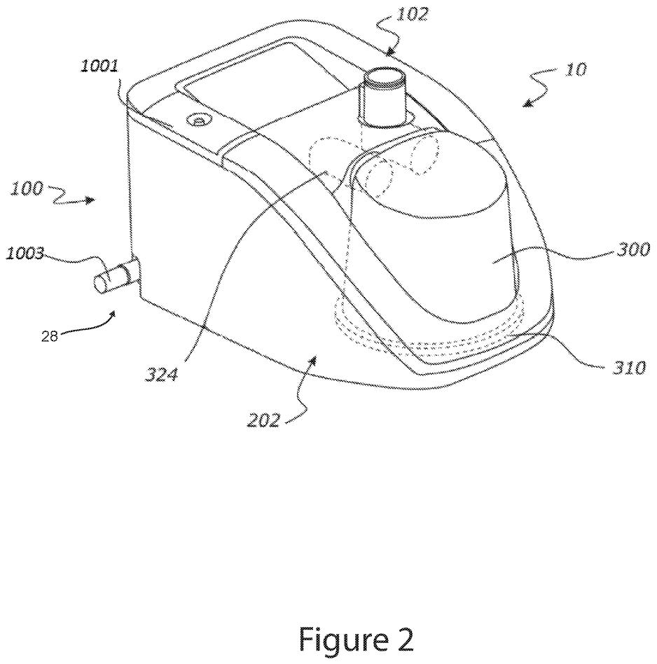

The humidifier 12 may comprise a humidifier heater 310 for example as a heater plate (see FIG. 2). The humidifier heater provides heat to the humidification chamber 300. The liquid in the humidification chamber may be water or another liquid, and/or may comprise a mixture of one or more liquids (for example a mixture of water and a medicament.)

The heater 310 of the humidifier 12 may be an electrically conductive heating element.

The controller 13 can be configured or programmed to control the operation of the breathing assistance apparatus 10. For example, the controller 13 can control components of the breathing assistance apparatus 10, including but not limited to: operating the flow generator 11 to create a flow of gas (gases flow) for delivery to a patient, operating the humidifier 12 (if present) to humidify and/or heat the generated gases flow, controlling a flow of oxygen into the flow generator blower, receiving user input from the user interface 14 for reconfiguration and/or user-defined operation of the breathing assistance apparatus 10, and outputting information (for example on the display) to the user.

The controller 13 may comprise one or more sub-controllers. The sub-controllers may each be configured to control one or more components of the apparatus (for example a flow generator sub-controller, and/or a humidifier sub-controller and/or a humidifier or conduit heater sub-controller). The controller 13 may include a master controller configured to communicate with, and pass commands to, the sub-controllers.

The controller 13 may include one or more computer processors and associated non-transitory memory or storage mediums storing processor-executable instructions or code. The instructions, when executed by the one or more processors cause the respiratory therapy apparatus to affect the steps and processes described herein.

It will be appreciated that when the specification describes the apparatus 10 undertaking an action, it may be that the controller 13 is controlling one or more components of the apparatus 10 to undertake the action.

It will be appreciated the methods described herein can be executed by the controller (or another processor).

The term breathing assistance apparatus may be used interchangeably with respiratory assistance apparatus, or respiratory therapy apparatus or flow therapy apparatus.

The term breathing assistance system may be used interchangeably with respiratory assistance system, or respiratory therapy system, or flow therapy system.

The term current flow rate may refer to a measurement of a flow rate which has been presently made (for example at a current time step). It will be appreciated that the term current flow rate is not limited to the latest flow rate determination and could include recently made flow rate determinations (for example from a previous time step or the most recent flow rate determination), and/or a filtered flow rate determination made based on a series of past measurements (which may optionally include signal filtering and/or processing).

The methods described herein may be embodied as software or a software module as part of control software (for example computer-readable instructions) that are stored in the controller (or associated memory) and executed by the controller (and/or an associated processor).

In the context of receiving therapy the user is a patient, however in the context of interacting with the apparatus (for example interacting with a user interface) the user can be one or more of a patient, healthcare professional (for example a clinician), or anyone else interested in using the apparatus.

As used herein, a “gases flow” can refer to any flow of gases that may be provided by the breathing assistance apparatus, such as a flow of ambient air, a flow comprising substantially 100% oxygen, a flow comprising some combination of ambient air and oxygen, and/or the like.

A breathing conduit 16 is coupled at one end to a gases outlet 21 in the housing 100 of the breathing assistance apparatus 10. The breathing conduit 16 is coupled at another end to a patient interface 17 such as a non-sealed nasal cannula with a manifold 19 and nasal prongs 18. Additionally, or alternatively, the breathing conduit 16 can be coupled to a face mask, a nasal mask, a nasal pillows mask, an endotracheal tube, a tracheostomy interface, and/or the like.

A breathable conduit may be provided between the breathing conduit 16 and the patient interface 17.

In some embodiments, a different conduit type may be connected to the gases outlet 21, for example a disinfection conduit for use in the disinfection mode.

The gases flow that is generated by the breathing assistance apparatus 10 may be humidified and delivered to the patient via the breathing conduit 16 and the patient interface 17.

The breathing conduit 16 can have a heater 16a to heat the gases flow passing through to the patient. The heater 16a can be under the control of the controller 13. In at least one configuration, the heater 16a is a heater wire. The breathing conduit 16 and/or patient interface 17 can be considered part of the breathing assistance therapy system. The breathing assistance system 1 may comprise the breathing assistance apparatus 10, breathing conduit 16, and patient interface 17.

The heater 16a of the breathing conduit may be located:
a) in a lumen of the breathing conduit 16,
b) within the wall of the breathing conduit 16,
c) embedded in a wall of the breathing conduit 16,
d) embedded in a bead which forms the breathing conduit 16, the bead being configured to provide structural support to the conduit 16, or
e) located on an external surface of the breathing conduit 16, or
f) any one or combination of a)-e).

The heater 16a may extend linearly along the conduit 16, or be helically wrapped around the conduit or be helically wrapped within the conduit wall or interior.

The heater 16a of the breathing conduit 16 may be an electrically conductive heating element (for example a heater wire).

The controller 13 can control the flow generator 11 to generate a gases flow at the desired flow rate (for example a therapy flow rate). The controller 13 can also control a supplemental oxygen inlet to allow for delivery of supplemental oxygen. As described in more detail below the controller 13 may control an proportional valve positioned at the supplemental oxygen inlet to vary delivery of supplemental oxygen to the flow generator 11.

The controller 13 can also control a humidifier heater in the humidifier 12 and/or the heater 16a in the breathing conduit 16 to heat the gas to a desired temperature for a desired level of therapy and/or level of comfort for the patient.

The controller 13 can be provided with or can determine a suitable target temperature of the gases flow. The controller 13 may control the humidifier heater 12 and/or the heater 16a of the breathing conduit based on one or more suitable target temperature(s) of the gases flow.

The heater 16a of the breathing conduit 16 may be controlled by the controller 13 to reach a desired temperature. The desired temperature may be, or be based on, one or more temperature set points, and/or one or more humidity set points (for example a therapy humidity).

The humidifier heater 12 may be controlled by the controller 13 to reach a desired temperature. The desired temperature may be, or be based on, one or more temperature set points, and/or one or more humidity set points. The desired temperature may be a therapy parameter.

The controller 13 may control the heater 16a of the breathing conduit 16 and/or the humidifier heater 12 to the desired temperature by closed loop control based on the output of one or more sensors.

The one or more temperature set points may relate to one or more therapy parameters of the apparatus for therapy (for example, a dew point or temperature of the gases), or be provided in the memory of the apparatus (for example, a predetermined temperature).

The apparatus may provide a plurality of therapy modes. The therapy modes may be manually selected by a user, or automatically, and once selected the apparatus may provide the selected therapy mode to the patient.

The apparatus may provide any one or combination of: Nasal High Flow (NHF) therapy, Continuous Positive Airway Pressure (CPAP) therapy, Non-Invasive Ventilation (NIV) and Bubble Continuous Positive Airway Pressure (BCPAP) therapy. The apparatus may comprise one or more control modes associated with each therapy type. The control modes may be manually selected by the user or automatically selected depending on the components connected to the apparatus (for example, dependent on the type of tube and/or patient interface connected to the apparatus). Each control mode may have an associated control scheme for controlling components of the apparatus (for example the flow generator, humidifier heater or conduit heater 16a).

The one or more therapy parameters for NHF therapy may comprise any one or combination of:
a therapy flow rate of the gases provided to the user,
a therapy humidity level (for example, a relative or absolute humidity, or a dew point),
a therapy oxygen concentration provided to the user,
a therapy concentration of an auxiliary gas provided to the user,
a therapy temperature of the gases provided to the user (for example a high flow rate).

The one or more therapy parameters for BCPAP therapy may comprise any one or combination of:
a therapy flow rate of the gases provided to the user,
a therapy humidity level (for example, a relative or absolute humidity, or a dew point),
a therapy oxygen concentration provided to the user,
a therapy concentration of an auxiliary gas provided to the user,
a therapy temperature of the gases provided to the user.

The one or more therapy parameters for CPAP therapy may comprise any one or combination of:
a therapy humidity level (for example a relative or absolute humidity, or a dew point)
a therapy oxygen concentration provided to the user,
a therapy temperature of the gases provided to the user,
a therapy concentration of an auxiliary gas provided to the user,
a therapy level of pressure support (for example, a CPAP pressure) provided to the user,
a therapy PEEP pressure provided to the user.

NIV therapy may be bi-level pressure therapy, where a first pressure is provided during an inspiration phase of a patient (i.e. a inspiratory airway pressure) and a second pressure is provided during an expiration phase of a patient (i.e. an expiratory airway pressure).

The one or more therapy parameters for Bi-level therapy, i.e., NIV therapy may comprise any one or combination of:
a therapy humidity level (for example, a relative or absolute humidity, or a dew point),
a therapy oxygen concentration provided to the user,
a therapy temperature of the gases provided to the user,
a therapy concentration of an auxiliary gas provided to the user,
a therapy IPAP pressure (inspiratory positive airway pressure) provided to the user,
a therapy EPAP pressure (expiratory positive airway pressure) provided to the user.

The therapy temperature may comprise a therapy temperature at the chamber outlet and/or a therapy temperature at the end of the breathing conduit.

The therapy humidity may be at the chamber outlet or at the end of the breathing conduit.

The therapy humidity level may be a dew point of about 27 degrees Celsius to about 40 degrees Celsius, or about 29 degrees Celsius to about 39 degrees Celsius, or about 31 degrees Celsius to about 38 degrees Celsius, or about 37 degrees Celsius, and/or an absolute humidity of above about 38 mgH2O or about to 44 mgH2O.

Providing humidity to a user may increase patient comfort during and compliance with therapy. The provision of humidity also provides additional benefits such as improving mucus transport, which is useful in patients with obstructive pulmonary diseases (e.g., COPD).

The user may enter one or more therapy parameters via the user interface of the breathing assistance apparatus.

The desired temperatures may be as measured at end of the breathing conduit 16, at the patient interface, at the gases outlet, a humidification chamber outlet, at any other sensor(s) of the apparatus, and/or any combination thereof.

The one or more temperature set points may comprise one or more of:
a desired dew point (for example, a temperature indicative of a desired humidity),
a predetermined dew point,
a predetermined temperature,
a desired temperature.

In some embodiments, the controller 13 may control the heater 16a of the breathing conduit 16 based on a desired temperature of the gases at the patient interface and/or a desired temperature at the end of the breathing conduit 16.

The apparatus may be powered by a mains power supply. For example, via a wired connection with an electrical grid. Alternatively, a source of power at a mains power supply level may be, for example, a portable electrical generator, distributed generation source, and/or a non-portable electrical generator such as a hospital back-up generator.

In some embodiments, the mains power supply may be any power supply configured to be connected to the apparatus via the electrical socket 114.

In some embodiments, the mains power supply may include any power supply which does not have a capacity constraint (for example, as with a battery which will deplete its energy storage over time when drawn from).

The apparatus may be powered by a non-peak power limited supply or a peak power limited supply (for example, a battery which may have a maximum rated current and/or power output below a peak current and/or power rating of the apparatus). Peak power (or current) limited in this context refers to the peak power required by the apparatus to operate with full capability.

The apparatus may be powered by an integrated power supply or an external power supply.

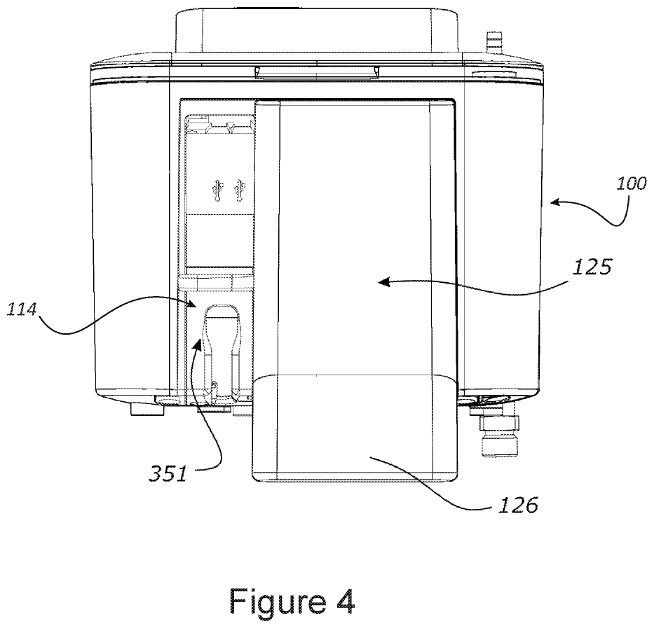

In some embodiments, for example as shown in FIG. 4, the apparatus may comprise at least one battery as part of a battery module 125 (with optional battery cover 126). The battery module may be located within the housing of the apparatus, and/or attached externally to the housing of the apparatus (as shown in FIG. 4). In some embodiments, the battery is removable (as shown in FIG. 4) and optionally connectable and disconnectable from the apparatus 10. Alternatively, the battery is non-removable.

The battery 125 may comprise a plurality of cells that provide voltage and current (i.e., power).

The term ‘battery’ as used herein is intended to encompass battery modules, battery packs, and other similar devices that may incorporate multiple batteries and/or battery cells and, optionally, additional hardware such as battery management systems (BMS), monitors, communications modules, etc. The term battery may be used interchangeably in the specification with the above terms.

The battery (for example battery module 125) may comprise a battery detect pin and/or a battery detect port. The battery detect pin and/or a battery detect port may be configured to communicate with the apparatus that the battery is connected. The battery detect pin may comprises a pull-up or pull-down resistor connected to the battery detect pin.

The battery module may comprise one or more memory elements, the one or more memory elements configured to store one or more battery parameters.

The battery parameters may comprise:
a) the battery expiry date,
b) battery cell states,
c) battery charge states,
d) the number of charge and discharge cycles the battery has experienced,
e) battery capacity (for example, as a percentage of an original maximum capacity), or
f) any one or combination of a)-e).

The apparatus 10 may comprise an electrical socket 114 configured to receive a plug of a power cord connected to the apparatus to provide power to the apparatus from a mains power supply.

The power cord plug removably connects to the electrical socket 114, such that if the power cord becomes damaged during use it can be replaced without having to perform any rewiring of the apparatus 10.

The apparatus 10 may comprise a power cord retainer 351 as shown in FIG. 4. The power cord retainer 351 may connect to a battery cover 126 of the battery module 125, such that during assembly the power cord would be attached to the apparatus 10 after the battery cover 126, with the power cord retainer 351 being attached last. Alternatively, the power cord retainer 351 could be connected to a different part of the housing 100.

The apparatus may comprise a battery charger configured to charge the battery 125. The apparatus may be configured to charge the battery when the apparatus is powered by a mains power supply (and optionally when the apparatus detects that a mains voltage is present for example when a voltage of the mains power supply is at or above a mains power supply threshold for example 110V or 230V +/−10 %) and the battery is not fully charged (and optionally when the battery charge is below a charge threshold for example 95%).

The battery module may communicate 13 one or more battery parameters as described above to the controller.

In some embodiments, the battery may communicate to the controller 13 to request charging by the battery charger until the battery 125 is fully charged. In some embodiments, the battery module 125 comprises a battery monitor which monitors one or more battery parameters.

The apparatus 10 may comprise a mains power supply conversion circuit.

The mains power supply conversion circuitry may comprise one or more switched-mode power supplies (or for example any other AC to DC converters).

The mains power supply conversion circuit may be configured to convert the mains power supply to a low DC voltage (optionally about 3 Volts DC to about 60 Volts DC).

The apparatus 10 may comprise a battery conversion circuit. The battery conversion circuit configured to convert the battery supply to a low DC voltage (optionally about 3 Volts DC to about 60 Volts DC).

As shown for example in FIG. 2, the oxygen inlet port 28 includes a valve 1003 through which a pressurized gas may enter the respiratory therapy apparatus 10. The valve can control a flow of oxygen into the respiratory therapy apparatus 10. The valve can be any type of valve, including a proportional valve or a binary valve. The proportional valve may be an electronic proportional valve.

The source of oxygen can be an oxygen tank or a hospital oxygen supply. Medical grade oxygen is typically between 95% and 100% purity. Oxygen sources of lower purity can also be used. Examples of valve modules and filters are disclosed in U.S. Provisional Application No. 62/409,543, titled “Valve Modules and Filter”, filed on Oct. 18, 2016, and U.S. Provisional Application No. 62/488,841, titled “Valve Modules and Filter”, filed on Apr. 23, 2017, which are hereby incorporated by reference in their entireties.

The breathing assistance apparatus 10 can measure and control the oxygen content of the gas being delivered to the patient, and therefore the oxygen content of the gas inspired by the patient.

The breathing assistance apparatus 10 may provide high flow therapy, in which the high flow rate of gas delivered meets or exceeds the peak inspiratory demand of the patient.

Operation sensors 3a, 3b, 3c, such as flow, temperature, humidity, and/or pressure sensors can be placed in various locations in the breathing assistance apparatus 10. Additional sensors (for example, sensors 20, 25) may be placed in various locations on the breathing conduit 16 and/or patient interface 17 (for example, there may be a temperature sensor 29 at or near the end of the inspiratory conduit).

The respiratory therapy apparatus 10 may have a communications module 15 to enable the controller 13 to receive signals 8 from the sensors and/or to control the various components of the breathing assistance apparatus 10, including but not limited to the flow generator 11, humidifier 12, heater 16a, humidifier heater, or accessories or peripherals associated with the breathing assistance apparatus 10. Additionally, or alternatively, the communications module 15 may deliver data to a remote server or enable remote control of the respiratory therapy apparatus 10 or respiratory therapy system 1.

The communications module may comprise a transmitter, receiver and/or transceiver.

The communications module 15 may act as a network interface (for example as a modem).

The communications module 15 may use one or more communication protocols known in the art, for example Wi-Fi, Bluetooth, Zigbee, cellular (3G, 4G, or 5G etc).

The communications module 15 may allow for communication between the apparatus and a mobile device (for example a phone or a tablet via Bluetooth or Wifi).

The communications module may comprise a number of separate transmitters, receivers and/or transceiver for each, or for a group of communication protocol(s).

The communications module 15 may be configured to transmit data and receive data from one or more devices (for example a server) as described in more detail below.

In some embodiments, one or more leak or blockage events, or alarms (as described in more detail below) may be transmitted to one or more servers and/or devices (for example a computer, phone or tablet). Additional information (for example the time, duration, or severity) associated with the event or alarm may be additionally transmitted to the server and/or device.

As described above, the breathing assistance apparatus 10 can measure and control the oxygen content of the gas being delivered to the patient. Oxygen may be measured by placing one or more gas composition sensors (such as an ultrasonic transducer system) after the oxygen and ambient air have been mixed. The measurement can be taken within the respiratory therapy apparatus 10, the patient breathing conduit 16, the patient interface 17, or at any other suitable location.

The oxygen concentration measured in the apparatus may be equivalent to the fraction of delivered oxygen (FdO2) and may be substantially the same as the oxygen concentration the patient is breathing, the fraction of inspired oxygen (FiO2), and as such the terms may be seen as equivalent, for example during a therapy with a sealed interface (i.e. NIV and CPAP) and/or during high flow therapy, when the flow rate of gases delivered meets or exceeds the peak inspiratory demand of the patient, so as to minimise or prevent entrainment of ambient air.

Oxygen concentration may also be measured by using flow rate sensors on at least two of: the ambient air inlet conduit, the oxygen inlet conduit, and the patient breathing conduit, to determine the flow rate of at least two gases. By determining the flow rate of both inlet gases or one inlet gas and one total flow rate, along with the assumed or measured oxygen concentrations of the inlet gases (about 20.9% for ambient air, about 100% for oxygen), the oxygen concentration of the final gas composition can be calculated. Alternatively, flow rate sensors can be placed at all three of the ambient air inlet conduit, the oxygen inlet conduit, and the breathing conduit, to allow for redundancy and testing that each sensor is working correctly by checking for consistency of readings. Other methods of measuring the oxygen concentration delivered by the breathing assistance apparatus 10 can also be used.

The breathing assistance apparatus 10 can include a patient sensor 26, such as a pulse oximeter or a patient monitoring system, to measure one or more physiological parameters of the patient, such as a patient's blood oxygen concentration (for example blood oxygen saturation (SpO2)), heart rate, respiratory rate, perfusion index, and provide a measure of signal quality. The sensor 26 can communicate with the controller 13 through a wired connection or by communication through a wireless transmitter on the sensor 26. The sensor 26 may be a disposable adhesive sensor designed to be connected to a patient's finger. The sensor 26 may be a non-disposable sensor (i.e., a re-useable sensor). Sensors that are designed for different age groups and to be connected to different locations on the patient are available and can be used with the breathing assistance system 1. The pulse oximeter can be attached to the patient, typically at their finger, although other places such as an earlobe are also an option. The pulse oximeter can be connected to a processor in the respiratory therapy apparatus 10 and can constantly provide signals indicative of the patient's blood oxygen saturation. The patient sensor 26 can be a hot-swappable device, which can be attached or interchanged during operation of the breathing assistance apparatus 10. For example, the patient sensor 26 may connect to the breathing assistance apparatus 10 using a USB interface or using wireless communication protocols (such as Bluetooth®).

When the patient sensor 26 is disconnected during operation, the breathing assistance apparatus 10 may continue to operate in its previous state of operation for a defined time period. After the defined time period, the breathing assistance apparatus 10 may trigger an alarm, transition from automatic mode to manual mode, and/or exit control mode (e.g., automatic mode or manual mode) entirely. The patient sensor 26 may be a bedside monitoring system or other patient monitoring system that communicates with the breathing assistance apparatus 10 through a physical or wireless interface.

The breathing assistance apparatus 10 may comprise or be in the form of a high flow therapy apparatus.

In some examples, the breathing assistance apparatus 10 may be configured to at least, or only, provide high flow.

High flow therapy as discussed herein is intended to be given its typical ordinary meaning as understood by a person of skill in the art which generally refers to a breathing assistance apparatus delivering a targeted flow of humidified respiratory gases via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen litres per minute to about sixty litres per minute or greater. Typical flow rates for paediatric patients (such as neonates, infants and children) often range from, but are not limited to, about one litre per minute per kilogram of patient weight to about three litres per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names.

For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient, ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may, in some configurations, deliver gases to the patient at a flow rate of between about 1 LPM and about 100 LPM, or at a flow rate in any of the sub-ranges outlined above. Gases delivered may comprise a percentage of oxygen. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%.

High flow therapy may be effective in meeting or exceeding the patient's inspiratory flow, increasing oxygenation of the patient, and/or reducing the work of breathing.

High flow therapy may be administered to the nares of a patient and/or orally, or via a tracheostomy interface.

High flow therapy may generate a flushing effect in the nasopharynx such that the anatomical dead space of the upper airways is flushed by the high incoming gases flow. This can create a reservoir of fresh gas available for each and every breath, while reducing re-breathing of nitrogen and carbon dioxide. Meeting inspiratory demand and flushing the airways is additionally important when trying to control the patient's FdO2. High flow therapy can be delivered with a non-sealing patient interface such as, for example, a nasal cannula. High flow therapy may slow down respiratory rate of the patient. High flow therapy may provide expiratory resistance to a patient.

High flow therapy may be used to treat patients with obstructive pulmonary conditions e.g., COPD, bronchiectasis, dyspnea, cystic fibrosis, emphysema and/or patients with respiratory distress or hypercapnic patients.

The term “non-sealing patient interface” (i.e., unsealed patient interface) as used herein can refer to an interface providing a pneumatic link between an airway of a patient and a gases flow source (such as from flow generator 11) that does not completely occlude the airway of the patient. A non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or both nares of the patient and/or their mouth. For a nasal cannula the airway is through the nares.

In some embodiments, the “non-sealing patient interface” may comprise a tracheal interface.

CPAP therapy may comprise providing gases to a user at a continuous positive pressure (and optionally one or more therapy parameters as described in more detail above).

BCPAP therapy may comprise providing gases to a user at a therapy flow rate (and optionally one or more therapy parameters as described in more detail above).

Bilevel therapy may comprise providing gases to a user at a therapy IPAP and EPAP (and optionally one or more therapy parameters as described in more detail above).

A sealed interface, may be used when the apparatus is providing CPAP, Bilevel or BCPAP therapy.

The flow generator 11 can be or comprises a blower module. The blower module may comprise at least one blower 11 configured to generate said flow of gases.

The flow generator 11 can include an ambient air inlet port 27 through which ambient room air can be entrained into the blower. The breathing assistance apparatus 10 may also include an oxygen inlet port 28 leading to a valve through which a pressurized gas may enter the flow generator 11. The valve can control a flow of oxygen into the flow generator 11. The valve can be any type of valve, including a proportional valve or a binary valve.

The blower 11 can operate at a motor speed of greater than about 1,000 RPM and less than about 8,000 RPM, greater than about 2,000 RPM and less than about 10,000 RPM, or between any of the foregoing values. The blower 11 can mix the gases entering the blower 11 through the gas inlet (for example, the ambient air inlet port 27 and/or an oxygen inlet port 28). Using the blower 11 as the mixer can decrease the pressure drop relative to systems with separate mixers, such as static mixers comprising baffles.

The breathing assistance apparatus may further comprise a gas composition sensor. The gas composition sensor may be the sensor described below (for example the ultrasonic transducer configuration).

The breathing assistance apparatus 10 comprises a flow sensor. The flow sensor may be configured to measure a flow rate of the flow of breathable gas to a patient.

The controller 13 may comprise one or more processors. The processors may be configured with computer-readable instructions.

The controller 13 may comprise at least one memory element. The memory element may be configured to store said computer-readable instructions.

The memory element may be non-transitory computer readable medium.

The controller 13 may be a microprocessor or an ASIC, FPGA or a combination of ICs or microprocessors or other suitable components and/or architectures.

The breathing assistance apparatus may comprise at least one display module, configured to display an alarm output.

The breathing assistance apparatus may comprise at least one audible module configured to emit an audible alarm.

In some embodiments the at least one audible module may comprise a speaker.

The display module may comprise at least one display (for example a liquid crystal display (LCD), or a light emitting diode (LED) display, although it will be appreciated any display technology may be used).

The display module may be configured to receive inputs to the system (for example as a touch screen) and therefore be at least part, or display part of the user interface 14.

The display module may be configured to be an input/output (I/O) module. For example, the display module may be configured to receive inputs from a user and provide outputs to a user (for example as part of, or to display part of the user interface 14).

The display module may communicate with the controller 13. In some embodiments the display module may provide information to the controller 13 (for example set points). In some embodiments the display module may receive information from the controller 13 (for example alarms, sensor outputs, and/or other calculated variables.)

Some examples of flow therapy apparatuses are disclosed in International Application No. PCT/NZ2016/050193, titled “Flow Path Sensing for Flow Therapy Apparatus”, filed on Dec. 2, 2016, and International Application No. PCT/IB2016/053761, titled “Breathing Assistance Apparatus”, filed on Jun. 24, 2016, which are hereby incorporated by reference in their entireties.

As shown for example in FIG. 2, the breathing assistance apparatus comprises a housing 100. The housing 100 has a housing upper chassis 102 and a housing lower chassis 202.

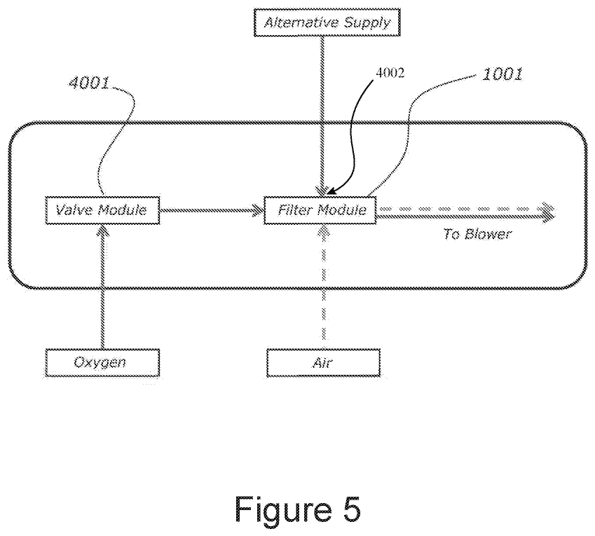

For example, as shown in FIG. 5, the apparatus 10 may comprise a valve module that controls the flow of oxygen and/or other gases entering the gas flow path of the apparatus 10 and enables the apparatus 10 to regulate the proportion of oxygen entrained in the airflow. The valve module is formed as a modular unit for ease of manufacture, assembly, servicing, or replacement. For example, in the event of malfunction, routine maintenance, or future upgrade/improvement.

The valve module may be configured to operate to control the oxygen concentration of the gases provided to the user to at a therapy oxygen concentration.

The apparatus 10 may comprise a filter module 1001, which may comprise a filter.

The filter modules and valve modules described herein may provide varying gas flow paths for the apparatus. For example, the valve module may control the flow of oxygen entering the gas flow path of the apparatus, via the valve module and filter module. Alternatively, the valve module may be bypassed by means of direct connection of an alternative oxygen source to the filter module via an alternative supply inlet. This may be practical in circumstances where a user may wish to manually adjust the oxygen supply (e.g., by a wall-supply rotameter).

It will be appreciated that the filter modules and the valve modules described herein may be used separately in apparatuses for delivering a flow of gas. Alternatively, the filter and the valve module may be used together as a filter and valve assembly for improved functionality.

In the configurations shown, the apparatus 10 receives oxygen by at least one of the following:
via the valve module (for automatic oxygen regulation by the apparatus), or
via the alternative gases inlet provided on the top of the filter (allowing attachment of a manually adjustable oxygen supply-such as a wall supply regulated by a regulator).

In some embodiments, the alternative gases inlet may be provided with a therapeutic gas that is not oxygen (for example heliox).

The apparatus 10 may comprise manifold. The manifold may be located on the housing. The manifold may provide one or more of: the oxygen inlet, the alternative gases inlet, and/or the air inlet.

The manifold may provide the oxygen, alternative gases, and/or ambient air to the valve module, filter module, and/or the blower.

The manifold may be provided upstream of the blower.

The oxygen inlet or alternative gasses supply inlet may be provided on a side of the manifold.

The manifold may allow excess oxygen to overflow to the ambient environment, and/or may allow oxygen to overflow to the ambient environment if the blower is off and oxygen is continually supplied. This prevents accumulation of concentrated oxygen in the housing of the apparatus.

The manifold may comprise one or more baffles that help to mix the oxygen and/or the alternative gases and air.

The manifold may also comprise a filter configured to filter the oxygen and/or the alternative gases and/or air from the respective inlets.

The various configurations described are exemplary configurations only. Any one or more features from any of the configurations may be used in combination with any one or more features from any of the other configurations.

In some embodiments, a motor and/or a sensor sub-assembly (for example containing one or more sensors) may be located in the apparatus housing.

The motor and/or sensor sub-assembly may be located in a recess on or in the underside of the apparatus housing. The recess may alternatively be in the rear, side, front, or top of the housing. The air and/or oxygen inlets may also be positioned differently as required.

As another example, rather than the humidification chamber and chamber bay being configured so that the humidification chamber is inserted into and removed from the chamber bay from a front of the housing, the configuration could be such that the humidification chamber is inserted into and removed from the chamber bay from a side, rear, or top of the housing.

As another example, while the filter modules are described as being inserted into the housing from above and the valve modules inserted into the housing from below, either or both of those components could be inserted into any suitable part of the housing, such as an upper part, lower part, side part, front part, or rear part.

The filter module and valve module are described with reference to a breathing assistance apparatus that can deliver heated and humidified gases to a patient or user.

The filter module and/or valve module may alternatively be used with an apparatus that does not require a humidifier and therefore does not require the humidification chamber 300. For example, it will be appreciated that the configuration that isolates the motor and gas flow path from the electrical and electronic components has broad applications in other types of gas delivery apparatuses.

As described above, the system may be provided with a battery, battery pack, or battery module 125. The battery may be provided as part of the apparatus as described above, or as part of the system more generally.

Reducing the charge level of batteries/battery pack(s) may be important in some cases. For example: in shipping, long term storage, or in the case of a recall, reducing the charge level of the batteries/battery pack(s) may minimise risk of damage to the batteries/battery pack and/or the apparatus as whole and increase safety for users and other personnel.

Reducing the charge level of the battery may be undertaken manually by a user, but this may require the attention of a user and may lead to less accurate charge levels (for example, the user may undershoot or overshoot the desired charge level). Undershooting a desired charge level may present a safety or equipment risk, while overshooting could result in an excessively deep discharge that affects the lifespan of the battery, for example.

Further, manual reduction of the charge level of the battery may lead to safety issues if components of the apparatus are controlled by the controller as per one or more therapy modes. For example, if the user selects a therapy mode of the apparatus in an attempt to manually reduce the charge level of the battery, the apparatus may control components to provide therapy as per the therapy mode. This may lead to safety issues if the apparatus isn't safely located or supervised closely. Further, if a chamber is connected, humidification may be provided to the conduits of the system and parts of the device which may present issues in storage, as residual moisture may remain present in the system.

Other manual methods of reducing the charge level for example discharging the battery through an electrical load (for example, a resistive load) might present electrical safety risks for users and would require an electrical load be available.

If the battery is damaged it may also be unsafe for a user to discharge the battery using these manual methods.

The battery may be configured to supply power to at least one component of the apparatus.

The at least one component may be any of the components as described elsewhere in the specification.

The at least one component may comprise one or more of the following:
a heater of a humidifier,
a flow generator,
a heater of a conduit.

The controller of the apparatus may be configured to control the at least one component of the apparatus to discharge the battery to a predetermined charge level.

The term ‘charge level’ is used herein to describe an amount of energy and/or charge of the battery. However, it will be appreciated that the term ‘charge level’ could be replaced with any other variable which describes the charge level of the battery, and/or relates to the amount of energy remaining in the battery.

It will be appreciated that when it is described that the battery is discharged to a charge level it may be discharged to a predetermined charge level within a tolerance (for example +/−5%).

In some configurations, the predetermined charge level is indicative of a state of charge of the battery.

In some configurations the predetermined charge level may be, or may be based on, an electrical characteristic. For example, it may be a voltage such as the terminal voltage. The electrical characteristic may be indicative of the state of charge of the battery.

In some configurations the predetermined charge level may be a predetermined state of charge. That is, the controller may be configured to control discharge of a state of charge of the battery to a predetermined state of charge.

The state of charge may be for example a real charge level with 0% being no charge and 100% being full charge or may be the state of charge presented to a user with, for example, 0% being 10% real charge and 100% being 90% real charge.

The controller may be configured to control a charge level of the battery to the predetermined charge level.

The charge level may be indicative of a state of charge of the battery. The charge level may additionally or alternatively be based on more or other battery characteristics.

The controller may ascertain a determined charge level of the battery and use the determined charge level to control discharge of the battery to the predetermined charge level.

The charge level (for example, the state of charge of the battery) may be based on one or more of: an electrical characteristic of the battery, a chemical characteristic of the battery, and/or a physical characteristic of the battery.

The determined charge level (for example, the state of charge of the battery) may be configured to be continuously, or periodically updated.

In some configurations, the controller may be in communication with the battery. For example, the controller may determine the characteristics from the battery directly. The controller may be configured to determine one or more of:
a state of charge of the battery,
an electrical characteristic of the battery,
a chemical characteristic of the battery, and/or
a physical characteristic of the battery.

In some configurations, alternatively or additionally, the controller may be provided with (for example, from another controller optionally associated with the battery) one or more of:
a state of charge of the battery,
an electrical characteristic of the battery,
a chemical characteristic of the battery, and/or
a physical characteristic of the battery.

The battery or battery pack may comprise a battery controller in communication with the controller of the apparatus.

The battery controller may be provided separate from the controller (i.e., in a battery pack housing), or could be part of the controller of the apparatus.

In some configurations, the battery controller may be configured to determine one or more of:
a state of charge of the battery,
an electrical characteristic of the battery,
a chemical characteristic of the battery, and/or
a physical characteristic of the battery.

The predetermined charge level may be based on, at least in part, a battery discharge configuration.

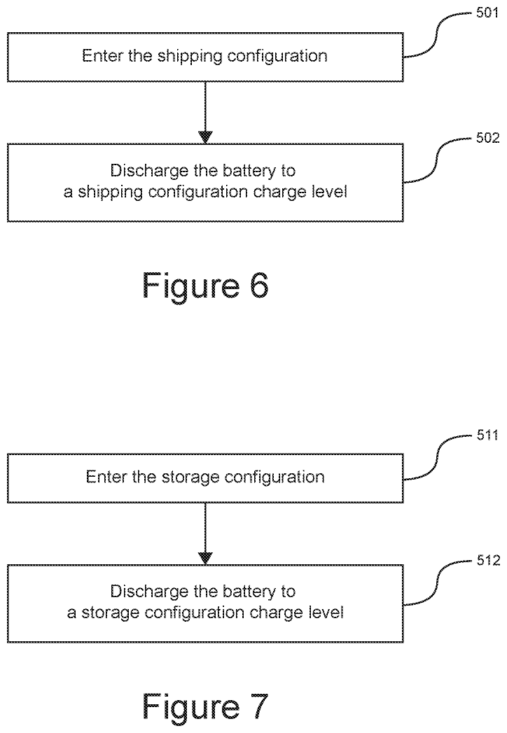

In some configurations, the battery discharge configuration may comprise a shipping configuration. In the shipping configuration, the predetermined charge level is a shipping configuration charge level. The shipping configuration charge level may be about a 30% state of charge. For example, as shown in FIG. 6, at step 501, the apparatus first enters the shipping configuration, then at step 502 the controller discharges the battery to a shipping configuration charge level.

In some configurations, the battery discharge configuration may comprise a storage configuration. In some configurations of the storage configuration the predetermined charge level is a storage configuration charge level. The storage configuration charge level may be about a 60% state of charge. For example, as shown in FIG. 7, at step 511 the apparatus first enters the storage configuration, then at step 512 the controller discharges the battery to a storage configuration charge level.

In some configurations of the storage configuration, the predetermined charge level may be based on the intended duration of storage. For example, when the intended duration of storage is about 6 months to about 12 months, the predetermined charge level may be a first storage configuration charge level (optionally about a 30% state of charge). By way of further example, when the intended duration of storage is greater than about 12 months, the predetermined charge level may be a second storage configuration charge level (optionally about a 60% state of charge).

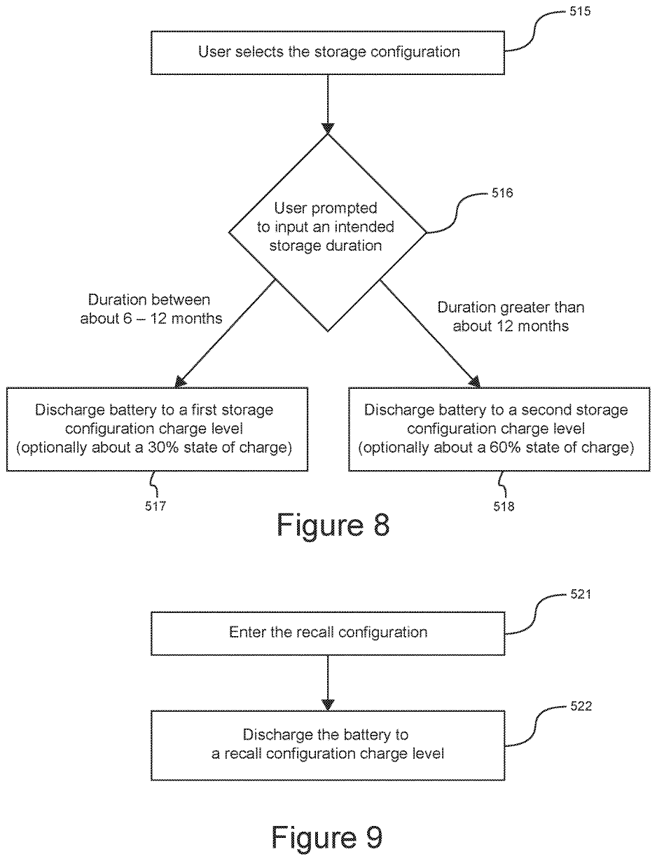

A user may enter the intended storage duration via a user interface. In some configurations, when a user selects the storage configuration, the apparatus may prompt the user (for example, by a display) to enter an intended storage duration via a user interface. For example, as shown in FIG. 8, after the user selects the storage configuration at step 515, the user is prompted to input an intended storage duration at step 516; if the user enters that the duration of storage is between about 6 months to about 12 months, the controller discharges the battery to the first storage configuration charge level (optionally about a 30% state of charge) at step 517; if the user enters that the duration of storage is greater than about 12 months, then the controller discharges the battery to the second storage configuration charge level (optionally about a 60% state of charge) at step 518.

In some configurations, the battery discharge configuration may comprise a recall configuration. In some configurations of the recall configuration the predetermined charge level may be recall configuration charge level (optionally the recall configuration charge level is a 10% state of charge). In the recall configuration the battery may be discharged to below recall configuration charge level. For example, as shown in FIG. 9, at step 521, the user enters or selects the recall configuration, then at step 522 the controller discharges the battery to the recall configuration charge level (optionally about a 10% state of charge, or below about a 10% state of charge).

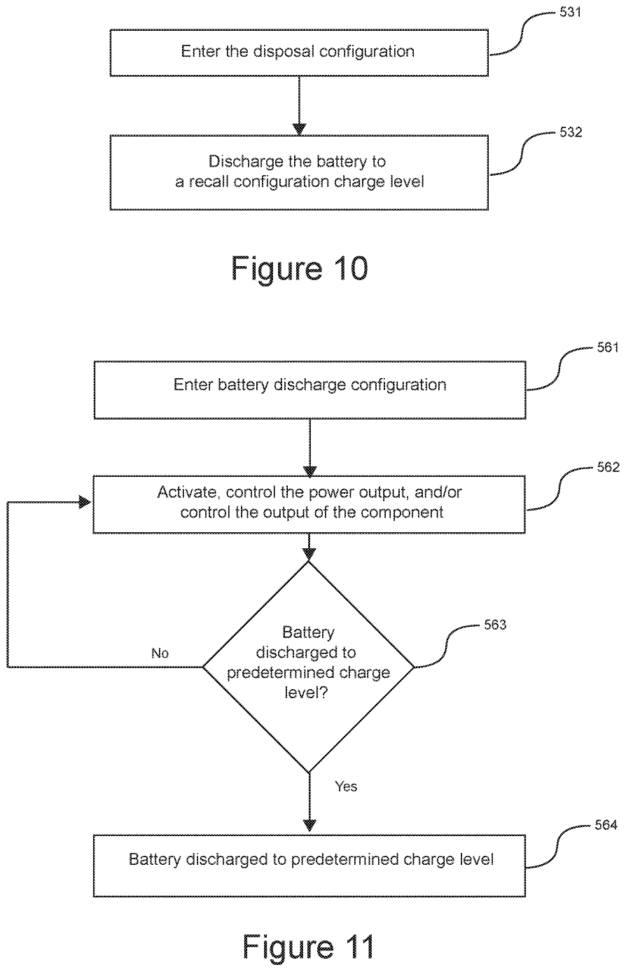

In some configurations the battery discharge configuration may comprise a disposal configuration. In some configurations of the disposal configuration the predetermined charge level is a disposal configuration charge level. The disposal configuration charge level may be about a 0% state of charge. For example, as shown in FIG. 10, at step 531, the user enters or selects the disposal configuration, then at step 532 the controller discharges a state of charge of the battery to a disposal configuration charge level (optionally about a 0% state of charge).

The controller may be configured to control the at least one component of the apparatus to discharge the battery at a predetermined discharge rate. In some configurations, the discharge rate may be a C-rate. The C-rate can be defined as the battery discharge current normalised to the total capacity of the battery.

In some configurations, the predetermined discharge rate may be based on the battery discharge configuration.

Controlling the at least one component may comprise one or more of:
activating and/or deactivating the at least one component
controlling a power draw of the at least one component, and/or
controlling an output of the at least one component.

In some configurations, controlling the at least one component may be based on the battery discharge configuration. That is, for different battery discharge configurations the controller may control the components in different ways.

When the battery discharge configuration is a storage configuration or a shipping configuration, the predetermined discharge rate may be a first discharge rate. When the battery discharge configuration is a recall configuration or a disposal configuration, the predetermined discharge rate may be a second discharge rate. In some configurations, the second discharge rate may be lower (i.e., slower) than the first discharge rate. That is, in such configurations, the predetermined discharge rate in the storage and/or shipping configurations may be higher than the discharge rate in the recall and/or disposal configurations.

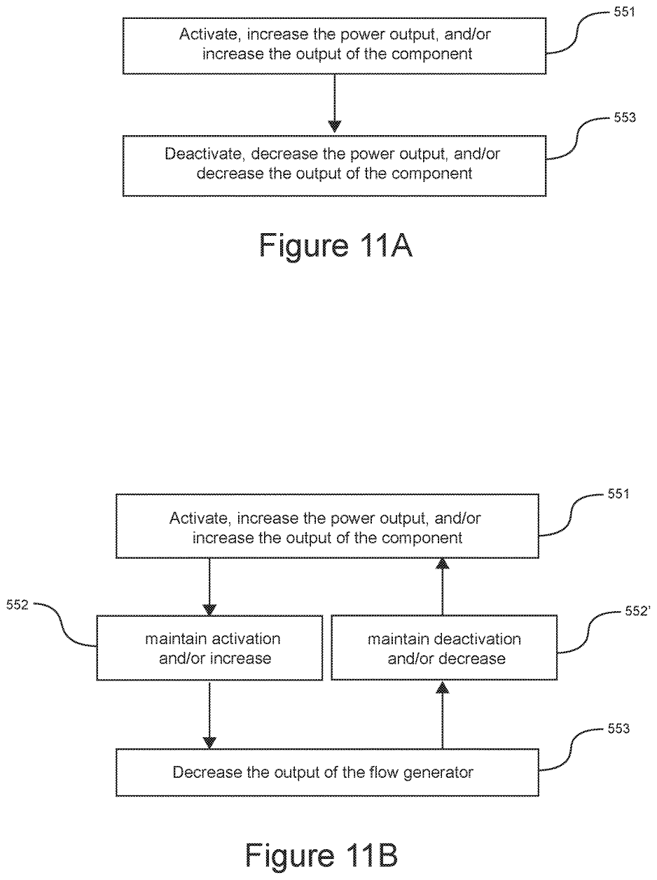

FIG. 11 shows an example where a battery discharge configuration is entered at step 561. At step 562, the controller activates at least one component, controls the power output to (i.e. power supplied to) the at least one component, and/or controls the output of the component. At step 563, if the battery has discharged to the predetermined charge level for the particular mode, then the battery is discharged to the predetermined charge level of the battery discharge configuration at step 564. If not, the controller continues to control the component at step 562.

As shown in FIGS. 11A and 11B, in some configurations the controller may be configured to activate the component, increase the power draw of the component, and/or increase the output of the component at step 551, and then deactivate the component, decrease the power draw of the component, and/or decrease the output of the component at step 553. Optionally, the controller may undertake the activating and/or increasing and deactivating and/or decreasing repeatedly.

Activating and/or increasing and deactivating and/or decreasing repeatedly the component may consume more energy (and hence discharge the battery faster) than maintaining activation of the component. For example, when the component is a motor, the accelerating and decelerating repeatedly the motor may consume more energy than running the motor at a constant speed. Similarly, increasing and then decreasing power output to the heater plate and/or heater of the breathing conduit repeatedly may also consume more energy than maintain a constant power output.

Optionally, as shown in step 552 in FIG. 11B, after the controller activates the component, increases the power draw of the component, and/or increases the output of the component the controller may be configured to maintain the output of the component (optionally for a predetermined amount of time).

Optionally, as shown in step 552′ in FIG. 11B, after the controller deactivates the component, decreases the power draw of the component, and/or decreases the output of the component the controller may be configured to maintain the output of the component (optionally for a predetermined amount of time).

The flow generator may comprise a blower. In some configurations the blower may include a motor.

In some configurations, the controller may be configured to control an output of the flow generator to discharge the battery to a predetermined charge level. The output of the flow generator may be one or more of: a speed of the motor, a flow rate of gases provided by the flow generator, and/or a pressure provided by the flow generator.

In some configurations, the controller may be configured to control an output of the flow generator to:
achieve a predetermined output of the flow generator, or
be within a predetermined output range of the flow generator, or
be above a predetermined output of the flow generator.

The predetermined output of the flow generator may be greater than a therapeutic output of the flow generator, the therapeutic output being an output when the apparatus is providing therapy to a user.

The predetermined output range of the flow generator may be a motor speed range of about 5,000 RPM to about 20,000 RPM, or about 23,000 RPM to about 26,000 RPM. For example, the predetermined output may be a motor speed of about 20,000 RPM.

When the apparatus 10 is configured to provide high flow therapy the flow generator output (for example motor speed) may be higher than for example a CPAP apparatus, and therefore the apparatus which provides high flow therapy may discharge the battery at a faster rate relative to a CPAP apparatus. The flow generator output (for example, motor speed) may be higher during provision of high flow therapy due to the need for high flow therapy to provide gases at a high flow rate through an unsealed interface, while during CPAP therapy the flow generator controls pressure to a sealed interface, and so does not need to provide gases at a high flow rate. For example, CPAP devices may not have power electronics and/or motor designs suitable to provide higher flows and/or higher motor speeds (such as those associated with high flow therapy).

The controller may be configured to control an output of the flow generator to repeatedly (and optionally, periodically), increase and decrease the output of the flow generator.

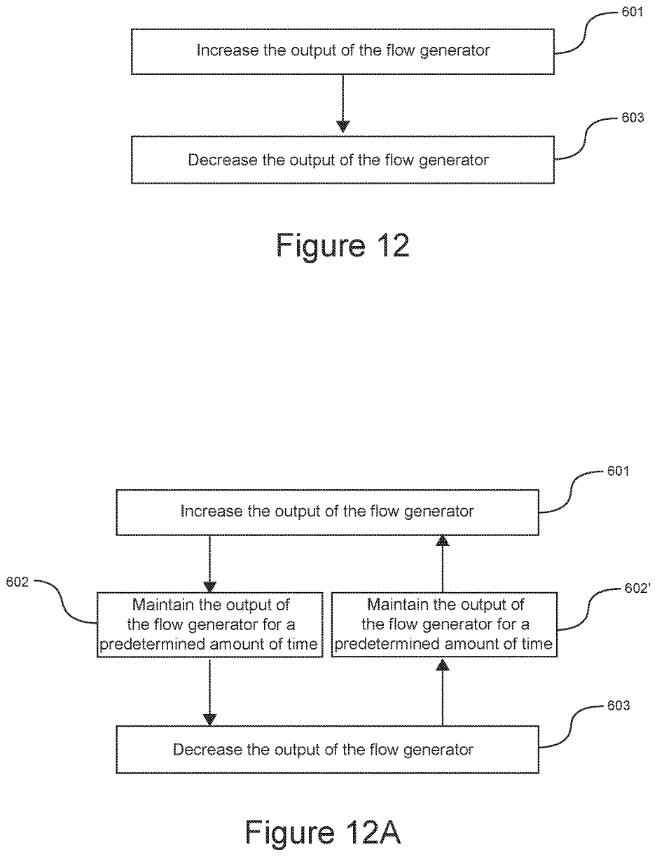

For example, as shown in FIG. 12, at step 601, the controller increases the output of the flow generator, before subsequently decreasing the output of the flow generator at step 603. The process may be undertaken repeatedly returning to step 601 after step 603. Optionally, the controller may maintain the output of the flow generator (optionally, for a predetermined amount of time) between steps 601 and 603 as shown in more detail below.

By way of further example, as shown in FIG. 12A, at step 601, the controller increases the output of the flow generator. Subsequent to the controller increasing the output of the flow generator, the controller is configured to then maintain the output of the flow generator for a predetermined amount of time at step 602, before subsequently decreasing the output of the flow generator at step 603. Subsequent to the controller decreasing the output of the flow generator, the controller is configured to maintain the output of the flow generator for a predetermined amount of time at step 602′. The predetermined amount of time at step 602 may be the same or different to that in step 602′. The predetermined amount of time at step 602 and predetermined amount of time at step 602′ may be based on the battery discharge mode.

In some configurations, the controller may control the output of the flow generator to increase and decrease within the predetermined output range of the flow generator.

In some configurations the controller may be configured to control the output of the flow generator to increase the output of the flow generator to a:
first predetermined flow generator output,
be within a first predetermined flow generator output range, or
be above a first predetermined flow generator output, and

subsequently decrease the output of the flow generator to:
a second predetermined flow generator output,
be within a second predetermined flow generator output range, or
be below a second predetermined flow generator output.

Decreasing the output of the flow generator may comprise allowing the motor of the flow generator to freewheel, or it may comprise controlling the output of the flow generator to decrease.

In some configurations the flow generator may comprise a braking system configured to decrease the output of the flow generator. The braking system may brake the motor of the flow generator; for example, the braking system may operate to decrease the motor speed.

The controller may be configured to control a power delivered to (for example provided to) the humidifier heater and/or the conduit heater. In some configurations, the controller may control the power delivered to the humidifier heater and/or the conduit heater by regulating a current or voltage delivered to the humidifier heater and/or the conduit heater. In some configurations, the controller may control the power delivered to the humidifier heater and/or the conduit heater by a digital control scheme. For example, the controller may be configured to control the humidifier heater and/or the conduit heater via pulse width modulation. In some configurations, the controller may be configured to provide a signal to the humidifier heater and/or the conduit heater to control the power delivered to the humidifier heater and/or the conduit heater.

The controller may be configured to control the power delivered to the humidifier heater and/or the conduit heater to discharge the battery to a predetermined charge level. For example, the controller may be configured to control the power delivered to the humidifier heater and/or the conduit heater to achieve a predetermined output of the humidifier heater and/or the conduit heater. By way of further example, the controller may be configured to control the power delivered to the humidifier heater and/or the conduit heater to achieve a predetermined heater power, or be within a predetermined heater power range, or be above a predetermined heater power.

In some configurations, the predetermined power of the heater may be greater than a therapeutic power, the therapeutic power being a power of the heater when the apparatus is providing therapy to a user. In some configurations, the predetermined power of the heater may correspond with a safe threshold for a user. In some configurations, the predetermined power of the heater may be based at least in part on characteristics of the heater (for example, physical size, location, heater type (e.g., heating filaments or ceramic heating elements), and/or electrical characteristics such as the electrical ratings of the heater).

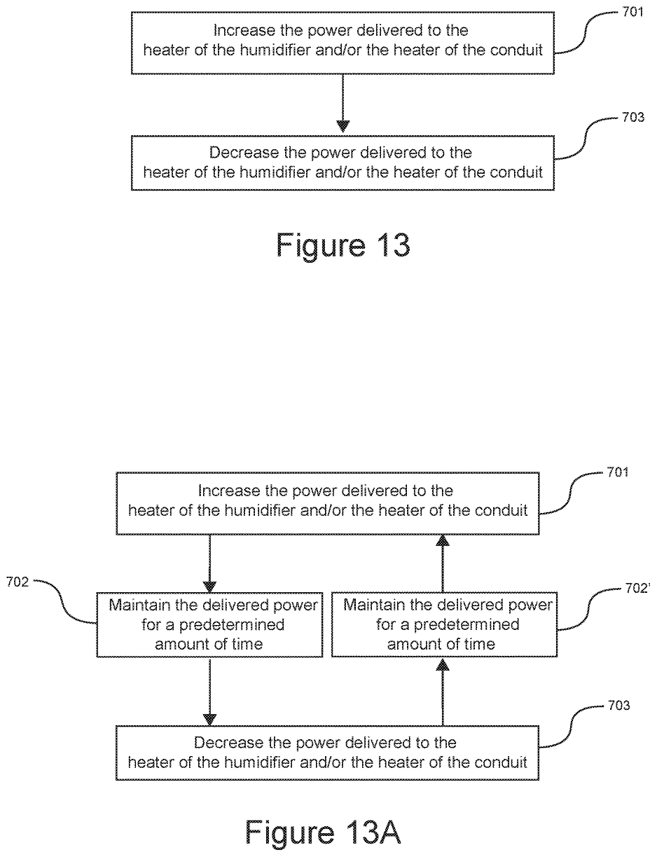

The controller may be configured to control the power delivered to the humidifier heater and/or the conduit heater to repeatedly and/or periodically increase and decrease. For example, as shown in FIG. 13, at step 701, the controller increases the power delivered to the humidifier heater and/or the conduit heater, before subsequently decreasing the power delivered to the humidifier heater and/or the conduit heater at step 703. The process may be undertaken repeatedly, for example, by after step 703, moving to step 701. Optionally, the controller may maintain the power delivered to the humidifier heater and/or the conduit heater (optionally for a predetermined amount of time) between steps 701 and 703 as shown in more detail below.

By way of further example, as shown in FIG. 12A, at step 701, the controller increases the power delivered to the humidifier heater and/or the conduit heater, subsequent to the controller increasing the power delivered to the humidifier heater and/or the conduit heater, the controller is configured to maintain the power delivered to the humidifier heater and/or the conduit heater for a predetermined amount of time at step 702, before subsequently decreasing the power delivered to the humidifier heater and/or the conduit heater at step 703. Subsequent to the controller decreasing the power delivered to the humidifier heater and/or the conduit heater, the controller is configured to maintain the power delivered to the humidifier heater and/or the conduit heater for a predetermined amount of time at step 702′. The predetermined amount of time at step 702 may be the same or different to that in step 702′. The predetermined amount of time at step 702 and predetermined amount of time at step 702′ may be based on the battery discharge mode.

In some configurations, the increase and decrease of the power delivered may be within a gas target temperature range and/or a humidity heater temperature range and/or a conduit heater temperature range.

The controller may be configured to control the power delivered to the humidifier heater and/or the conduit heater, to increase the power delivered to:
a first predetermined power delivered to the humidifier heater and/or a first predetermined power delivered conduit heater,
within a first predetermined power delivered to the humidifier heater range and/or within a first predetermined power delivered conduit heater range, or
above a first predetermined power delivered to the humidifier heater and/or a above a first predetermined power delivered conduit heater, and

subsequently decrease the power delivered to the humidifier heater and/or the power delivered conduit heater to:
a second predetermined power delivered to the humidifier heater and/or a second predetermined power delivered conduit heater,
within a second predetermined power delivered to the humidifier heater range and/or within a second predetermined power delivered conduit heater range, or
below a second predetermined power delivered to the humidifier heater and/or a below a second predetermined power delivered conduit heater.

The controller may be configured to control the power delivered to the humidifier heater and/or the conduit heater to achieve a target gas temperature. The target gas temperature may be a temperature of the gases as detected at one or more locations. The one or more locations may comprise:

### an Inlet of the Humidifier,

an outlet of the humidifier,
a location in the conduit, and/or
a location at the patient end of the conduit.

The apparatus may comprise at least one temperature sensor. The temperature sensor(s) may be configured to measure the temperature of the gases at the one or more locations.

In some configurations, the target gas temperature may be greater than a therapeutic temperature. The therapeutic temperature may be the gas temperature when the apparatus is providing therapy to a user. In some configurations, the target gas temperature may correspond with a safe threshold for a user.

In some configurations, the controller may be configured to control the power delivered to the humidifier heater and/or the conduit heater to achieve a target heater plate temperature. The target heater plate temperature may be for example less than about 50 degrees Celsius, or less than about 60 degrees Celsius, or less than about 70 degrees Celsius.

In some configurations, the controller may be configured to control the power delivered to the humidifier heater to achieve a target humidifier heater temperature range and/or control the power delivered to the conduit heater to achieve a target conduit heater temperature range, wherein the target humidifier heater temperature range and/or the target conduit heater temperature range is/are about 50 degrees Celsius to about 80 degrees Celsius, or about 50 degrees Celsius to about 70 degrees Celsius, or about 60 degrees Celsius to about 70 degrees Celsius.

The apparatus may comprise at least one heater temperature sensor. The at least one heater temperature sensor may be configured to measure the temperature of the humidifier heater and/or the conduit heater.

In some configurations, the heater temperature may be greater than a therapeutic heater temperature. The therapeutic temperature may be the heater temperature when the apparatus is providing therapy to a user. In some configurations, the heater temperature of the heater may correspond with a safe threshold for a user (for example, a safe threshold for touching the conduit surface or heater plate surface).

The controller may be configured to control the target gas temperature and/or the heater temperature to repeatedly and/or periodically increase and decrease.

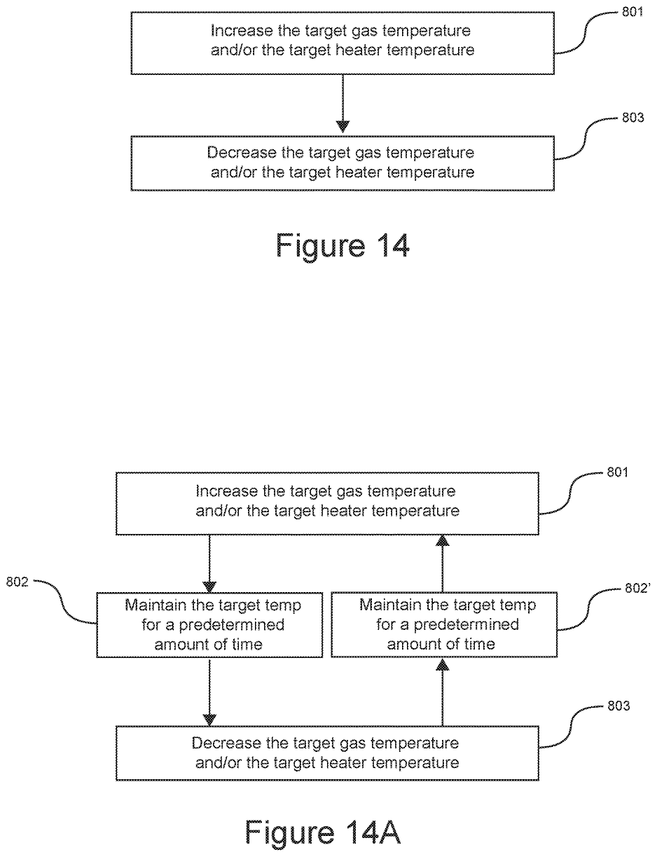

For example, as shown in FIG. 14, at step 801, the controller increases the target gas temperature and/or the target heater temperature, before subsequently decreasing the target gas temperature and/or the target heater temperature at step 803. The process may be undertaken repeatedly, for example, by after step 803, moving to step 801.

Optionally, the controller may maintain the target gas temperature and/or the target heater temperature (optionally for a predetermined amount of time) between steps 801 and 803 as shown in more detail below.

By way of further example, as shown in FIG. 14A, at step 801, the controller increases the target gas temperature and/or the target heater temperature, subsequent to the controller increasing the target gas temperature and/or the target heater temperature, the controller is configured to maintain the target gas temperature and/or the target heater temperature for a predetermined amount of time at step 802, before subsequently decreasing the target gas temperature and/or the target heater temperature at step 803. Subsequent to the controller decreasing the target gas temperature and/or the target heater temperature, the controller is configured to maintain the target gas temperature and/or the target heater temperature for a predetermined amount of time at step 802′. The predetermined amount of time at step 802 may be the same or different to that in step 802′. The predetermined amount of time at step 802 and predetermined amount of time at step 802′ may be based on the battery discharge mode.

In some configurations the increase and decrease of the target gas temperature and/or the target heater temperature may be within a gas target temperature range and/or the humidity heater temperature range and/or a conduit heater temperature range.

The increase and decrease of the target gas temperature and/or the target heater temperature may provide a safe way of discharging the battery. For example, if the temperatures are controlled within a range that is safe to a user (for example being safe to touch, at least briefly, without being burned), then safety of the apparatus during discharging can be guaranteed.

The controller may be configured to control the target gas temperature and/or the target heater temperature to increase to a:
first predetermined target gas temperature and/or first predetermined target heater temperature,
within a first predetermined target gas temperature range and/or within a first predetermined target humidity heater temperature range and/or within a first predetermined target conduit humidity heater temperature range, or
above a first target gas temperature and/or a first target heater temperature, and subsequently decrease the output of the humidifier heater and/or conduit heater to:
second predetermined target gas temperature and/or second predetermined target heater temperature,
within a second predetermined target gas temperature range and/or within a second predetermined target humidity heater temperature range and/or within a second predetermined target conduit humidity heater temperature range, or
below a second target gas temperature and/or a second target heater temperature.

The controller may be configured to control the output of the flow generator to increase the output of the flow generator to a:
first predetermined flow generator output,
within a first predetermined flow generator output range, or
above a first predetermined flow generator output, and subsequently decrease the output of the flow generator to:
a second predetermined flow generator output,
within a second predetermined flow generator output range, or
below a second predetermined flow generator output.

It will be appreciated that the various ways of discharging the battery above may be combined and used together in any combination. For example the steps shown in FIGS. 11A-14A could be undertaken sequentially of cyclically with other steps in the FIGS. 11A-14A.

It will be appreciated that various ways of discharging different components may be undertaken at the same time. For example, one or any combination of the heater of the humidifier, heater of the conduit and flow generator may be controlled simultaneously, or sequentially to discharge the battery.

The controller may be configured to power off the apparatus once the battery is discharged to a predetermined charge level.

If after the battery discharge process has been completed and the battery charge level is below the predetermined charge level (for example by more than an acceptable margin) the controller may charge the battery to the predetermined charge level. Charging the battery may mean the user has to connect the apparatus to a mains power source (and the apparatus may provide a prompt to remind the user to do so). Charging the battery may be undertaken at a normal charge rate, or at a reduced charge rate to avoid overshooting the predetermined charge level.

The apparatus may comprise at least one display. The display may be in communication with controller. The display may be part of the display module as described in more detail above. In some configurations, the controller may be configured to control the display to display a notification that the battery is being discharged. In some configurations, the controller may be configured to control the display to display the battery discharge configuration.

In some configurations, the display may also display that the battery is due for a replacement or recall. If the battery is due for replacement or recall, the display may also prompt the user to initiate the discharge process (for example to initiate the discharge of the battery to the predetermined charge level). In some configurations the apparatus may receive a message that the battery is due for replacement or recall from a remote device.

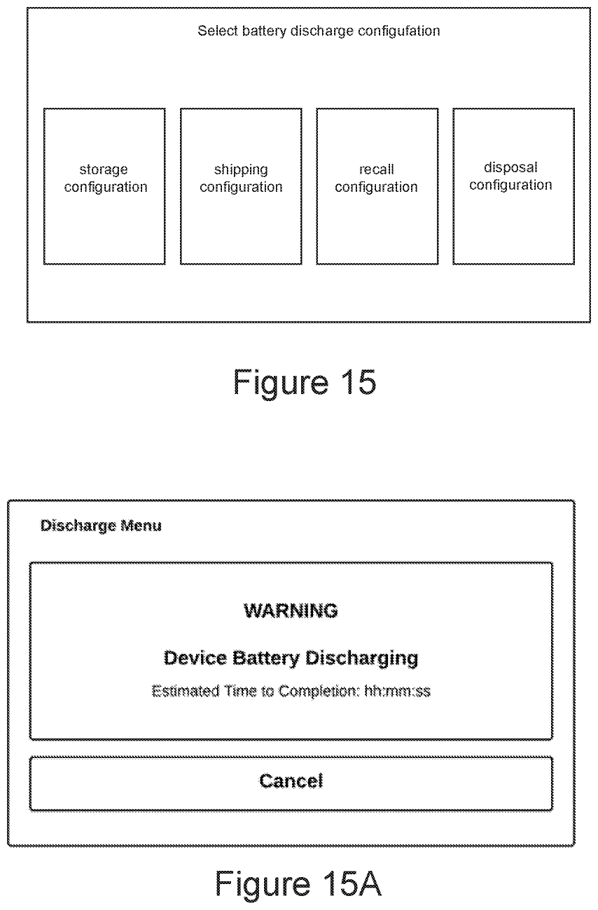

FIG. 15 shows an example of a display showing options for a user to select different battery discharge configuration.

FIG. 15A shows an example of a discharge menu shown on the display during the discharge process. The discharge menu may show a warning to the user telling them not to use the apparatus, as well as an indication that the battery is discharging.

The discharge menu may also comprise a cancel button to allow the process to be cancelled.

In some configurations, the apparatus may comprise a user interface. By way of example, the user interface may be a touch interface; the touch interface may be part of the display.

In some configurations, the apparatus may be configured to allow the user to initiate the discharge of the battery to a predetermined charge level via the user interface. In some configurations, the apparatus may further be configured to allow the user to initiate the discharge of the battery to the predetermined charge level based on one or more of:
the user providing credentials associated with permissions allowing the initiation of the battery discharging to a predetermined charge level; and/or
accessing a service menu, optionally via providing a sequence of inputs via the user interface.

In some configurations, the apparatus may be configured to allow the user to initiate the discharge of the battery to a predetermined charge level based on a message from a remote device.

The apparatus may not present the user interface allowing initiation of the battery discharge until a message is received by the apparatus from a device.

The message may be based on a serial number of the apparatus and/or a serial number of the battery.

In some configurations, the apparatus may be provided with a serial number list of batteries which for example may need to be serviced or recalled. The apparatus may then automatically enter the recall battery discharge configuration or prompt the user to enter the recall battery discharge configuration.

For example, the user may be provided with an option for the recall battery discharge configuration if the serial number of the battery matches a serial number list provided as part of the message from the device.

By way of further example, the apparatus may be provided with the shipping configuration, if the message from the device indicates that the apparatus is to be returned to the supplier.

In some configurations, a user can use the user interface to cancel the discharge of the battery. In some configurations, the display may comprise an interactable element configured to trigger the controller to cancel the discharge of the battery.

The apparatus may be configured to determine whether a patient is connected to the patient interface. In some configurations, the apparatus may determine whether a patient is connected to the user interface based on one or more of the following: a flow rate, a pressure data of the flow of gases, and/or a resistance to flow of the flow of gases.

In some configurations, when the apparatus determines that a patient is connected to the user interface, the controller may be configured to cancel the discharge of the battery.

The determination that a patient is connected may be that as described in WO/2020/178746 which is hereby incorporated by reference.

The humidifier may be configured to be pneumatically connected to the flow generator. In some configurations, the humidifier may be further configured to humidify the flow of gases generated by the flow generator.

The conduit may be connected to an outlet of the apparatus. The conduit may be one or more of: an expiratory conduit, an inspiratory conduit, and/or a patient supply conduit. In some configurations, the conduit may be pneumatically connected to the apparatus. In some configurations, the conduit may form part of a gases flow path.

In some configurations the conduit is a discharge conduit as described in more detail below.

The apparatus may comprise a heater plate as the heater. In some configurations, the heater plate may be configured to heat the contents of a humidification chamber.

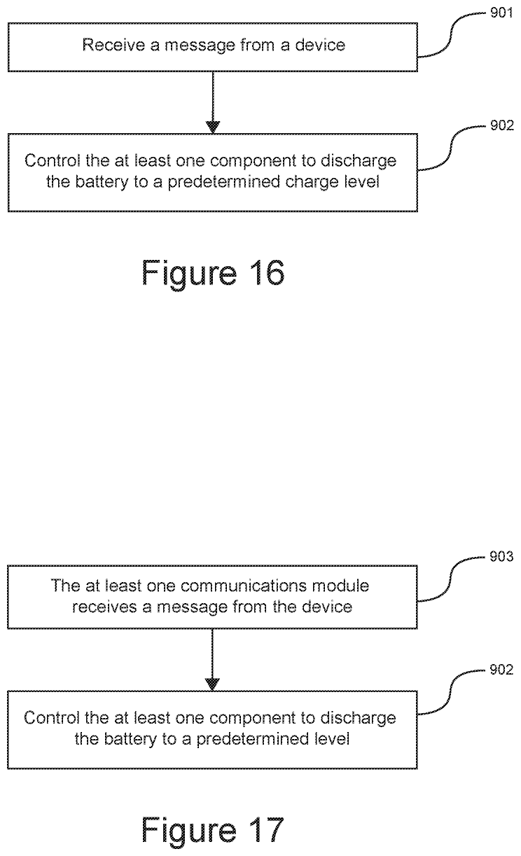

In some configurations, the apparatus may be configured to control the at least one component of the apparatus to discharge the battery to a predetermined charge level when the apparatus receives a message from a device. That is, as shown in FIG. 16, at step 901, the apparatus receives a message from a device, then after the apparatus receives the message, the controller controls the at least one component to discharge the battery to a predetermined charge level at step 902.

In some configurations, the apparatus may comprise at least one communications module configured to receive the message from the device.

As shown in FIG. 17, at step 920, the at least one communications module receives a message from the device, after the apparatus receives the message, the controller controls the at least one component to discharge the battery to a predetermined charge level at step 902.

The device may be for example one or more of a server, a computer, a phone, and/or a tablet. In some configurations, the device may be configured to select one of a plurality of connected apparatuses by a device ID associated with each apparatus.

In some configurations, the device may be configured to receive an input from a user interface, and based on the input, communicate the message to the apparatus. For example, the device may receive an input from a user interface of the device and communicates the message to the apparatus; upon receiving the message from the device, the controller then controls the at least one component of the apparatus to discharge the battery to a predetermined charge level.

In some configurations, the battery of the breathing assistance apparatus may be prepared by controlling the at least one component of the apparatus to discharge the battery to a predetermined charge level. The at least one component may for example comprise a heater of a humidifier, a flow generator, or a conduit configured to be connected to the apparatus, the conduit comprising a heater.

In some configurations, a discharge conduit may be provided for connection with the apparatus. The discharge conduit may be used specifically to discharge the battery.

The discharge conduit may for example use more power but generate less heat so as to safely dissipate more energy in a safer manner for example without generating excess heat or damaging the conduit. When the discharge conduit is used the humidifier heater may be disabled. The discharge conduit may comprise a resistance load and/or a higher resistance heater wire (relative to a normal conduit for use in therapy).

The discharge conduit may be connected from a humidifier outlet to a chamber inlet to form a loop for the gases to recirculate within the system.

If the discharge mode is connected to the apparatus therapy modes may be disabled.

Where in the foregoing description reference has been made to elements or integers having known equivalents, then such equivalents are included as if they were individually set forth.

When the apparatus is started and where that battery is in a battery charge configuration, the apparatus may present a message to the user. The message may show the battery charge configuration that the battery is currently in and/or the message may be a reminder to charge the battery.

When the apparatus is started after being in any one of a storage configuration, recall configuration or a shipping configuration, the message may be presented informing the user to charge the battery. In some configurations the apparatus may prevent use of the device until the battery is charged to a predetermined charge level (for example 70%, 80%, 90% or 100%).

The battery may be charged via the apparatus, when connected to the apparatus. The apparatus may comprise battery charging circuity configured to charge the battery. The battery charging circuity may be configured to convert the input voltage of the apparatus to a battery charging voltage. The battery charging circuity may also monitor parameters of the battery to determine charge level (as described above).

In some configurations, the battery may be able to be charged via a wired connection between the battery and a charger (for example via a USB connection via a USB port on the battery pack.) The charger may be the apparatus as described above or an external charger.

In some configurations, the battery may be charged by a battery charging station which the battery (and optionally a number of batteries) may be coupled to.

Although embodiments have been described with reference to a number of illustrative embodiments thereof, it will be understood by those skilled in the art that various changes in form and details may be made therein without departing from the spirit and scope of the invention as defined by the appended claims. Therefore, the preferred embodiments should be considered in a descriptive sense only and not for purposes of limitation, and also the technical scope of the invention is not limited to the embodiments. Furthermore, the present invention is defined not by the detailed description of the invention but by the appended claims, and all differences within the scope will be construed as being comprised in the present disclosure.

Many modifications will be apparent to those skilled in the art without departing from the scope of the present invention as herein described with reference to the accompanying drawings.

1. A breathing assistance apparatus comprising:
a battery configured to supply power to at least one component of the apparatus,
the at least one component comprising one or more of:
a humidifier heater,
a flow generator, or
a conduit, the conduit comprising a conduit heater, and
a controller configured to control the at least one component of the apparatus to discharge the battery to a predetermined charge level.

2. The breathing assistance apparatus of claim 1, wherein the controller is in communication with the battery, and wherein the controller is configured to determine one or more of: a state of charge of the battery, an electrical characteristic of the battery, a chemical characteristic of the battery and/or a physical characteristic of the battery.

3. The breathing assistance apparatus of claim 1, wherein the controller is provided with one or more of: a state of charge of the battery, an electrical characteristic of the battery, a chemical characteristic of the battery and/or a physical characteristic of the battery.

4. The breathing assistance apparatus of claim 1, wherein the battery comprises a battery controller in communication with the controller of the apparatus, and wherein the battery controller is configured to determine one or more of:
a state of charge of the battery,
an electrical characteristic of the battery,
a chemical characteristic of the battery and/or
a physical characteristic of the battery.

5. The breathing assistance apparatus of any one of the preceding claims, wherein the predetermined charge level is an electrical characteristic (optionally a voltage).

6. The breathing assistance apparatus of claim 5, wherein the electrical characteristic is indicative of a state of charge of the battery.

7. The breathing assistance apparatus of claims 1 to 4, wherein the predetermined charge level is indicative of a state of charge of the battery.

8. The breathing assistance apparatus of claims 1 to 4, wherein the predetermined charge level is a predetermined state of charge, and wherein the controller is configured control to discharge the battery to the predetermined state of charge.

9. The breathing assistance apparatus of any one of the preceding claims, wherein the predetermined charge level is based on, at least in part, at least one battery discharge configuration.

10. The breathing assistance apparatus of claim 9, wherein the at least one battery discharge configuration comprises a shipping configuration, wherein in the shipping configuration the predetermined charge level is a shipping configuration charge level.

11. The breathing assistance apparatus of claim 10, wherein the shipping configuration charge level is about a 30% state of charge.

12. The breathing assistance apparatus of claim 9, wherein the at least one battery discharge configuration comprises a storage configuration, wherein in the storage configuration the predetermined charge level is a storage configuration charge level.

13. The breathing assistance apparatus of claim 12, wherein the storage configuration charge level is about a 60% state of charge.

14. The breathing assistance apparatus of claim 12, wherein in the storage configuration the predetermined charge level is based on an intended duration of storage.

15. The breathing assistance apparatus of claim 14, wherein when the intended duration of storage is about 6 months to about 12 months, the predetermined charge level is a first storage configuration charge level.

16. The breathing assistance apparatus of claim 15, wherein the first storage configuration charge level is about a 30% state of charge.

17. The breathing assistance apparatus of claims 14 to 16, wherein when the intended duration of storage is greater than about 12 months, the predetermined charge level is a second storage configuration charge level.

18. The breathing assistance apparatus of claim 17, wherein the second storage configuration charge level is about a 60% state of charge.

19. The breathing assistance apparatus of claim 14 to 18, wherein a user can enter the intended storage duration via a user interface.

20. The breathing assistance apparatus of claim 14 to 18, wherein when a user selects the storage configuration, the apparatus prompts (optionally via a display) to enter the intended storage duration via a user interface.

21. The breathing assistance apparatus of claim 9, wherein the at least one battery discharge configuration comprises a recall configuration, wherein in the recall configuration the predetermined charge level is a recall configuration charge level.

22. The breathing assistance apparatus of claim 21, wherein the recall configuration charge level about a 10% state of charge.

23. The breathing assistance apparatus of claim 9, wherein the at least one battery discharge configuration comprises a disposal configuration, wherein in the disposal configuration the predetermined charge level is a disposal configuration charge level.

24. The breathing assistance apparatus of claim 23, wherein the disposal configuration charge level is about a 0% state of charge.

25. The breathing assistance apparatus of claims 9 to 24, wherein the controller is configured to control the at least one component of the apparatus to discharge the battery at a predetermined discharge rate.

26. The breathing assistance apparatus of claim 25, wherein the predetermined discharge rate is a C-rate.

27. The breathing assistance apparatus of claims 25 to 26, wherein the predetermined discharge rate is based on the battery discharge configuration.

28. The breathing assistance apparatus of claims 9 to 27, wherein when the at least one battery discharge configuration is a or the storage configuration or a or the shipping configuration the predetermined discharge rate is a first discharge rate.

29. The breathing assistance apparatus of claims 27 to 28, wherein when the at least one battery discharge configuration is a recall configuration or a disposal configuration the predetermined discharge rate is a second discharge rate.

30. The breathing assistance apparatus of claim 29, wherein the second discharge rate is lower than the first discharge rate.

31. The breathing assistance apparatus of any one of claims 1 to 30, wherein the controller is configured to control discharge of the battery to control a charge level of the battery to the predetermined charge level.

32. The breathing assistance apparatus of any one of claims 31, wherein the charge level is indicative of a state of charge of the battery.

33. The breathing assistance apparatus of claim 6, wherein the charge level of the battery is based on one or more of:
an electrical characteristic of the battery,
a chemical characteristic of the battery,
a physical characteristic of the battery.

34. The breathing assistance apparatus of claims 31 to 33, wherein a determined charge level indicative of the charge level of the battery is configured to be continuously, or periodically updated.

35. The breathing assistance apparatus of claims 9 to 30, wherein controlling the at least one component comprises one or more of:
activating and/or deactivating the at least one component
controlling a power draw of the at least one component,
controlling an output of the at least one component.

36. The breathing assistance apparatus of claim 35, wherein controlling the at least one component is based on the battery discharge configuration.

37. The breathing assistance apparatus of any preceding claim, wherein the controller is configured to:
activate the at least one component, increase the power draw of the at least one component, and/or increase the output of the at least one component, and then,
deactivate the at least one component, decrease the power draw of the at least one component, and/or decrease the output of the at least one component,
optionally the controller may undertake the activating and/or increasing and deactivating and/or decreasing repeatedly.

38. The breathing assistance apparatus of claim 37, wherein after the controller activates the at least one component, increases the power draw of the at least one component, and/or increases the output of the at least one component, the controller is configured to maintain the output of the at least one component (optionally for a predetermined amount of time).

39. The breathing assistance apparatus of claim 38 to 39, wherein after the controller deactivates the at least one component, decreases the power draw of the at least one component, and/or decreases the output of the at least one component the controller is configured to maintain the output of the at least one component (optionally for a predetermined amount of time).

40. The breathing assistance apparatus of any one of the preceding claims, wherein the controller is configured to control an output of the flow generator to discharge the battery to the predetermined charge level.

41. The breathing assistance apparatus of claim 40, wherein the flow generator comprises a blower, the blower including a motor.

42. The breathing assistance apparatus of claim 41, wherein the output of the flow generator is one or more of:
a speed of the motor,
a flow rate of gases provided by the flow generator,
a pressure provided by the flow generator.

43. The breathing assistance apparatus of claims 40 to 42, wherein the controller is configured to control the output of the flow generator to:
achieve a predetermined output of the flow generator, or
be within a predetermined output range of the flow generator, or
be above a predetermined output of the flow generator.

44. The breathing assistance apparatus of claim 43, wherein the predetermined output is greater than a therapeutic output of the flow generator, the therapeutic output being an output when the apparatus is providing therapy to a user.

45. The breathing assistance apparatus of claims 43 to 44, wherein the predetermined output range of the flow generator is a motor speed range of about 5,000 RPM to about 20,000 RPM, or about 23,000 RPM to about 26,000 RPM.

46. The breathing assistance apparatus of claim 43 to 45, wherein the predetermined output is a motor speed of about 20,000 RPM.

47. The breathing assistance apparatus of claims 40 to 42, wherein the controller is configured to control the output of the flow generator to repeatedly (optionally periodically) increase and decrease the output of the flow generator (optionally within a predetermined output range of the flow generator).

48. The breathing assistance apparatus of claim 47, wherein subsequent to the controller increasing the output of the flow generator, the controller is configured to maintain the output of the flow generator (optionally for a predetermined amount of time) before subsequently decreasing the output of the flow generator.

49. The breathing assistance apparatus of claims 47 to 48, wherein subsequent to the controller decreasing the output of the flow generator, the controller is configured to maintain the output of the flow generator (optionally for a predetermined amount of time) before subsequently increasing the output of the flow generator.

50. The breathing assistance apparatus of claims 47 to 49, wherein the controller is configured to control the output of the flow generator to increase the output of the flow generator to a:
first predetermined flow generator output,
be within a first predetermined flow generator output range, or
be above a first predetermined flow generator output, and
subsequently decrease the output of the flow generator to:
a second predetermined flow generator output,
be within a second predetermined flow generator output range, or
be below a second predetermined flow generator output.

51. The breathing assistance apparatus of claims 47 to 50, wherein decreasing the output of the flow generator comprises allowing the motor of the flow generator to freewheel or controlling the output of the flow generator to decrease.

52. The breathing assistance apparatus of claims 47 to 50, wherein decreasing the flow generator comprises a braking system configured to brake the motor of the flow generator.

53. The breathing assistance apparatus of claim 52, wherein the braking system is configured to decrease the output of the flow generator (optionally by decreasing the motor speed).

54. The breathing assistance apparatus of claims 1 to 53, wherein the controller is configured to control a power delivered to the humidifier heater and/or the conduit heater.

55. The breathing assistance apparatus of claim 54, wherein the power delivered to the humidifier heater and/or the conduit heater is controlled by regulating a current or voltage delivered to the humidifier heater and/or the conduit heater.

56. The breathing assistance apparatus of claim 54 or claim 55, wherein the power delivered to the humidifier heater and/or the conduit heater is controlled by a digital control scheme.

57. The breathing assistance apparatus of claim 56 wherein the controller is configured to control the humidifier heater and/or the conduit heater via pulse width modulation.

58. The breathing assistance apparatus of any one of claims 54 to 57, wherein the controller is configured to provide a signal to the humidifier heater and/or the conduit heater to control the power delivered to the humidifier heater and/or the conduit heater.

59. The breathing assistance apparatus of claims 54 to 58, wherein the controller is configured to control the power delivered to the humidifier heater and/or the conduit heater to discharge the battery to the predetermined charge level.

60. The breathing assistance apparatus of claims 54 to 59, wherein the controller is configured to control the power delivered to the humidifier heater and/or the conduit heater to achieve a predetermined output of the humidifier heater and/or the conduit heater.

61. The breathing assistance apparatus of claims 54 to 60, wherein the controller is configured to control the power delivered to the humidifier heater and/or the conduit heater to achieve a predetermined heater power, or be within a predetermined heater power range, or be above a predetermined heater power.

62. The breathing assistance apparatus of claim 61, wherein the predetermined heater power is greater than a therapeutic power, the therapeutic power being a power of the heater when the apparatus is providing therapy to a user.

63. The breathing assistance apparatus of claims 61 to 62, wherein the predetermined heater power corresponds with a safe threshold for a user.

64. The breathing assistance apparatus of claims 54 to 58, wherein the controller is configured to control the power delivered to the humidifier heater and/or the power delivered to the conduit heater to repeatedly (optionally periodically) increase and decrease the power delivered to the humidifier heater and/or the power delivered conduit heater (optionally within a gas target temperature range and/or a target humidifier heater temperature range and/or a target conduit heater temperature range).

65. The breathing assistance apparatus of claim 64, wherein subsequent to the controller increasing the power delivered to the humidifier heater and/or the power delivered to the conduit heater, the controller is configured to maintain the power delivered to the humidifier heater and/or the power delivered conduit heater before subsequently decreasing the power delivered to the humidifier heater and/or the power delivered to the conduit heater.

66. The breathing assistance apparatus of claims 64 to 65, wherein subsequent to the controller decreasing the power delivered to the humidifier heater and/or the power delivered to the conduit heater, the controller is configured to maintain the power delivered to the humidifier heater and/or the power delivered conduit heater (optionally for a predetermined amount of time) before subsequently increasing the power delivered to the humidifier heater and/or the power delivered to the conduit heater.

67. The breathing assistance apparatus of claims 64 to 66, wherein the controller is configured to control the power delivered to the humidifier heater and/or the power delivered to the conduit heater to increase the power delivered to the humidifier heater and/or the power delivered to the conduit heater to:
a first predetermined power delivered to the humidifier heater and/or a first predetermined power delivered to the conduit heater,
within a first predetermined power delivered to the humidifier heater range and/or within a first predetermined power delivered to the conduit heater range, or
above a first predetermined power delivered to the humidifier heater and/or above a first predetermined power delivered to the conduit heater, and
subsequently decrease the power delivered to the humidifier heater and/or the power delivered to the conduit heater to:
a second predetermined power delivered to the humidifier heater and/or a second predetermined power delivered to the conduit heater,
within a second predetermined power delivered to the humidifier heater range and/or within a second predetermined power delivered to the conduit heater range, or
below a second predetermined power delivered to the humidifier heater and/or a below a second predetermined power delivered to the conduit heater.

68. The breathing assistance apparatus of claims 1 to 62, wherein the controller is configured to control the power delivered to the humidifier heater and/or the conduit heater to achieve a target gas temperature, wherein the target gas temperature is a temperature of the gases at one or more of locations, the one or more locations comprising:
an inlet of the humidifier,
an outlet of the humidifier,
a location in the conduit,
a location at the patient end of the conduit.

69. The breathing assistance apparatus of claim 68, wherein the apparatus comprises at least one temperature sensor, the at least one temperature sensor configured to measure the temperature of the gases at the one or more locations.

70. The breathing assistance apparatus of claims 68 to 69, wherein the target gas temperature is greater than a therapeutic gas temperature when the apparatus is providing therapy to a user.

71. The breathing assistance apparatus of claims 68 to 70, wherein the target gas temperature corresponds with a safe threshold for a user.

72. The breathing assistance apparatus of any preceding claim, wherein the controller is configured to control the power delivered to the humidifier heater conduit to achieve a target humidifier heater temperature and/or to control the power delivered to the conduit heater to achieve a target conduit heater temperature, optionally the target humidifier heater temperature and/or the target conduit heater temperature is less than about 60 degrees Celsius.

73. The breathing assistance apparatus of any preceding claim, wherein the controller is configured to control the power delivered to the humidifier heater to achieve a target humidifier heater temperature range and/or to control power delivered to the conduit heater to achieve a target conduit heater temperature range, wherein the target humidifier heater temperature range and/or the target conduit heater temperature range is about 40 degrees Celsius to about 60 degrees Celsius.

74. The breathing assistance apparatus of claims 1 to 62 and 68 to 70, wherein the apparatus comprises at least one heater temperature sensor, the at least one heater temperature sensor configured to measure a temperature of the humidifier heater and/or a temperature conduit heater.

75. The breathing assistance apparatus of claim 74, wherein the temperature of the humidifier heater and/or the temperature of the conduit heater is/are greater than a therapeutic heater temperature when the apparatus is providing therapy to a user.

76. The breathing assistance apparatus of claims 74 to 75, wherein the temperature of the humidifier heater and/or the temperature of the conduit heater correspond(s) with a safe threshold for a user.

77. The breathing assistance apparatus of claims 68 to 76, wherein the controller is configured to control a or the target gas temperature and/or a or the target humidifier heater temperature and/or a or the target conduit heater temperature to repeatedly (optionally periodically) increase and decrease (optionally within a or the gas target temperature range and/or a or the target humidity heater temperature range and/or a or the target conduit heater temperature range).

78. The breathing assistance apparatus of claim 77, wherein subsequent to the controller increasing the target gas temperature and/or the target humidifier heater temperature and/or the target conduit heater temperature, the controller is configured to maintain the target gas temperature and/or the target humidifier heater temperature and/or the target conduit heater temperature before subsequently decreasing the target gas temperature and/or the target humidifier heater temperature and/or the target conduit heater temperature.

79. The breathing assistance apparatus of claims 77 to 78, wherein subsequent to the controller decreasing the target gas temperature and/or the target humidifier heater temperature and/or the target conduit heater temperature, the controller is configured to maintain the target gas temperature and/or the target humidifier heater temperature and/or the target conduit heater temperature (optionally for a predetermined amount of time) before subsequently increasing the target gas temperature and/or the target humidifier heater temperature and/or the target conduit heater temperature.

80. The breathing assistance apparatus of claims 77 to 79, wherein the controller is configured to control the target gas temperature and/or the target heater temperature to increase the target gas temperature and/or the target heater temperature to a:
first predetermined target gas temperature and/or first predetermined target humidifier heater temperature and/or a first predetermined target conduit heater temperature,
within a first predetermined target gas temperature range and/or within a first predetermined target humidifier heater temperature range and/or within a first predetermined target conduit heater temperature range, or
above a first target gas temperature and/or above a first target humidifier heater temperature and/or above a first target conduit heater temperature, and
subsequently decrease the output of the flow generator to:
a second predetermined target gas temperature and/or a second predetermined target humidifier heater temperature and/or a second predetermined target conduit heater temperature,
within a second predetermined target gas temperature range and/or within a second predetermined target humidifier heater temperature range and/or within a second predetermined target conduit heater temperature range, or
below a second target gas temperature and/or below a second target humidifier heater temperature and/or below a second target conduit heater temperature.

81. The breathing assistance apparatus of any preceding claim, wherein the controller is configured to power off the apparatus once the battery is discharged to a or the predetermined charge level.

82. The breathing assistance apparatus of any preceding claim, wherein the apparatus comprises at least one display, the display being in communication in with controller.

83. The breathing assistance apparatus of claim 82, wherein the controller is configured to control the at least one display to display a notification that the battery is being discharged.

84. The breathing assistance apparatus of claims 82 to 83, wherein the controller is configured to control the at least one display to display the battery discharge configuration.

85. The breathing assistance apparatus of any preceding claim, wherein the apparatus comprises a user interface (optionally the user interface is a touch interface as part of the at least one display).

86. The breathing assistance apparatus of claim 85, wherein a user can initiate the battery discharging to a or the predetermined charge level via the user interface.

87. The breathing assistance apparatus of any preceding claim, wherein the apparatus is configured to allow the user to initiate the battery discharging to a or the predetermined charge level based on one or more of:
the user providing credentials associated with permissions allowing the initiation of the battery discharging to a or the predetermined charge level;
accessing a service menu, optionally via providing a sequence of inputs via the user interface.

88. The breathing assistance apparatus of any preceding claim, wherein the apparatus is configured to allow the user to initiate the battery discharging to a or the predetermined charge level based on a message from a remote device.

89. The breathing assistance apparatus of claims 85 to 86, wherein a user can cancel the battery discharging via the user interface.

90. The breathing assistance apparatus of claims 82 to 85, wherein the at least one display comprises an interactable element configured to trigger the controller to cancel the battery discharging.

91. The breathing assistance apparatus of any preceding claim, wherein the apparatus is configured to determine whether a patient is connected to a patient interface.

92. The breathing assistance apparatus of claim 91, wherein the apparatus is configured to determine whether a patient is connected to the patient interface based on a flow rate and/or a pressure data of the flow of gases.

93. The breathing assistance apparatus of claim 91, wherein the apparatus is configured to determine whether a patient is connected to the patient interface based on a resistance to flow of the flow of gases.

94. The breathing assistance apparatus of claims 91 to 93, wherein when the apparatus determined a patient is connected to the patient interface, the controller is configured to cancel the battery discharging.

95. The breathing assistance apparatus of any preceding claim, wherein the humidifier is configured to be pneumatically connected to the flow generator and to humidify the flow of gases generated by the flow generator.

96. The breathing assistance apparatus of any preceding claim, wherein the conduit is connected to an outlet of the apparatus.

97. The breathing assistance apparatus of any preceding claim, wherein the conduit is one or more of: an expiratory conduit, an inspiratory conduit, a patient supply conduit.

98. The breathing assistance apparatus of any preceding claim, wherein the conduit is pneumatically connected to the apparatus.

99. The breathing assistance apparatus of any preceding claim, wherein the conduit forms part of a gases flow path.

100. The breathing assistance apparatus of any preceding claim, wherein the apparatus comprises a heater plate as the humidifier heater, wherein the heater plate is configured to heat the contents of a humidification chamber.

101. The breathing assistance apparatus of any preceding claim, wherein the controller is configured to control the at least one component of the apparatus to discharge the battery to a or the predetermined charge level when the apparatus receives a message from a device.

102. The breathing assistance apparatus of claim 101, wherein the apparatus comprises at least one communications module configured to receive the message from the device.

103. The breathing assistance apparatus of claims 101 to 102, wherein the device is a one or more of:
a server,
a computer,
a phone,
a tablet.

104. The breathing assistance apparatus of claims 101 to 103, wherein the device is configured to select one of a plurality of connected apparatuses by a device ID associated with each apparatus.

105. The breathing assistance apparatus of claims 101 to 104, wherein the device is configured to receive an input from a or the user interface and based on the input communicate the message to the apparatus.

106. A method of preparing a battery of a breathing assistance apparatus comprising:
controlling the at least one component of the apparatus to discharge the battery to a predetermined charge level, wherein the at least one component comprises:
a heater of a humidifier,
a flow generator,
a conduit configured to be connected to the apparatus, the conduit comprising a heater.

107. A breathing assistance system comprising:
a flow generator,
a humidifier configured to be pneumatically connected to the flow generator and to humidify a flow of breathable gases generated by the flow generator, the humidifier comprising at least one heater (optionally the heater is a heater plate),
a conduit configured to be powered by the apparatus, the conduit comprising a heater,
a controller configured to control one or more of: the flow generator, the humidifier heater, and/or the conduit heater, to discharge the battery to a predetermined charge level.

108. A breathing assistance apparatus comprising:
a battery configured to supply power to at least one component of the apparatus, the at least one component comprising one or more of:
a heater of a humidifier,
a flow generator,
a conduit configured to be powered by the apparatus, the conduit comprising a heater,
a controller configured to control the at least one component of the apparatus to discharge the battery to a predetermined charge level based on the battery discharge configuration,
the controller configured to receive a command relating to the selection of a battery discharge configuration from a user interface, wherein the user interface is part of the apparatus, or another device.
